(19) United States
(12) Patent Application Publication
(10) Pub. No.: US 2010/0168858 A1
(43) Pub. Date: Jul. 1, 2010

(54) Expandable interbody implant and method

(76) Inventors: Mitchell Hardenbrook, Hopkinton, MA (US); Joyce Lauer, Wayland, MA (US); Kevin Staid, Lowell, MA (US)

Correspondence Address: FRANCIS H. KIRKPATRICK;KIRKPATENT CONSULTING, 37 CLOVER HILL DR., CHELMSFORD, MA, 01824-2611, US

(21) Appl. No.: 12/655,425

(22) Filed: Dec. 30, 2009

Related U.S. Application Data
(63) Continuation-in-part of application No. 12/460,413, filed on Jul. 17, 2009.
(60) Provisional application No. 61/203,909, filed on Dec. 30, 2008.

(51) Int. Cl. A61F 2/44; A61B 17/58; A61B 17/70

(52) U.S. Cl. 623/17.12; 606/93; 623/17.16; 606/246

(57) ABSTRACT

An interbody implant system for use with a first vertebra having a first endplate and a second vertebra having a second endplate. The system includes an expandable implant that includes a plurality of supports and means for linking the plurality of supports. The plurality of supports are capable of moving apart from one another so that the expandable implant is in an expanded condition. A channel extends through the first vertebra from a pedicle or the body wall to the first endplate. An unexpanded diameter for the expandable implant is configured to permit passage of the expandable implant through the channel. An expanded diameter for the expandable implant is greater than the channel diameter at the first endplate. The support height is configured to permit the plurality of supports to be positioned between the first endplate and the second endplate. Methods for the use of the system are described.

### CROSS-REFERENCE TO RELATED APPLICATIONS

This application claims the benefit of the priority of U.S. Provisional Application No. 61/203,909, filed on Dec. 30 2008, and is a continuation-in-part of U.S. application Ser. No. 12/460,413, filed Jul. 17, 2009, each of which is incorporated by reference herein wherever permitted.

### BACKGROUND

The spine consists of a number of vertebrae, as well as spinal discs between the vertebrae that act as shock absorbers, and ligaments that link the vertebrae. The vertebrae, spinal discs, and ligaments, together with associated muscles, form a strong yet flexible column. Deterioration of vertebrae or spinal discs, or altered positioning of vertebrae, may result from various conditions, injuries, or disease states. Treatment of such deterioration or altered positioning may employ devices or methods that stabilize the position of a vertebra relative to one or more other vertebrae. Stabilization may employ surgical implantation of devices or prostheses. Stabilization may also include inducing new bone to grow between vertebrae, resulting in fusion of vertebrae.

Because of the high forces experienced by spinal components during normal movements, such as bending, a high degree of integrity is required of any devices that are provided for strengthening the spine or for correction of defects. Moreover, the devices must be implantable, and must be adapted to be inserted, aligned and adjusted from devices operating from outside the spinal column. Moreover, all components and all steps in a procedure on the spinal column must avoid damage to the nerves inside the spinal column or exiting through it. These goals are not easily met by an assortment of implants and implanting instruments, unless they are designed to work together to provide the desired final result.

### SUMMARY

The invention comprises implantable devices for correction of defects of the spinal column, and systems for their use. In one embodiment, the invention provides an interbody implant system for use with a first vertebra having a first endplate and a second vertebra having a second endplate. The system includes an expandable implant that includes a plurality of supports and means for linking the plurality of supports. The plurality of supports are capable of moving apart from one another so that the expandable implant is in an expanded condition. A channel extends through the first vertebra from a pedicle or the body wall to the first endplate. An unexpanded diameter for the expandable implant is configured to permit passage of the expandable implant through the channel. An expanded diameter for the expandable implant is greater than the channel diameter at the first endplate. The support height is configured to permit the plurality of supports to be positioned between the first endplate and the second endplate. Devices and methods for use of the invention are described and claimed.

### BRIEF DESCRIPTION OF THE DRAWINGS

FIG. 1 is a side view of four vertebrae in the lumbar and sacral regions of a human spine.

FIG. 2 is an axial cephalad view of vertebra L4 in the lumbar region of a human spine.

FIG. 3 is a partial section side view of an interbody implant system for use in a spine that includes a first vertebra and a second vertebra, the system comprising an expandable implant, the expandable implant comprising a plurality of supports and means for linking the plurality of supports.

FIG. 4 is a perspective view of the expandable implant of the system of FIG. 3 when the expandable implant is in an unexpanded condition.

FIG. 5 is a perspective view of the expandable implant of the system of FIG. 3 when the expandable implant is in an expanded condition.

FIG. 6 is a partial section side view of the system of FIG. 3, the view being taken during passage of the expandable implant through a channel in the first vertebra.

FIG. 7 is a section view of the expandable implant of the system of FIG. 3 when the expandable implant is in an unexpanded condition.

FIG. 8 is a section view of the expandable implant of the system of FIG. 3 when the expandable implant is in a partially expanded condition.

FIG. 9 is a section view of the expandable implant of the system of FIG. 3 when the expandable implant is in an expanded condition.

FIG. 10 is a section view of an expandable implant in which a passage for a guidewire is partially curved.

FIG. 11 is a partial section side view of an interbody implant system for use in a spine that includes a first vertebra and a second vertebra, the system comprising an expandable implant and means for expanding the expandable implant, in which the means for expanding comprises a wedge.

FIG. 12 is a partial section side view of an interbody implant system for use in a spine that includes a first vertebra and a second vertebra, the system comprising an expandable implant and means for expanding the expandable implant, in which the means for expanding comprises a wedge.

FIG. 13 is a section view of a means for expanding that comprises a wedge.

FIG. 14 is a perspective view of an expandable implant in which the means for linking comprises a sheet, the means for linking being attached at a peripheral surface or a lateral surface of the plurality of supports, the expandable implant being in an expanded condition.

FIG. 15 is a perspective view of an expandable implant in which the means for linking comprises a sheet, the means for linking being attached at a central surface or a lateral surface of the plurality of supports, the expandable implant being in an expanded condition.

FIG. 16 is a section view of an expandable implant in which the means for linking is attached at a central surface or a lateral surface of the plurality of supports, the expandable implant being in a partially expanded condition.

FIG. 17 is a section view of an expandable implant that includes a first means for linking that is attached at a central or lateral surface for the plurality of supports and a second means for linking that is attached at a peripheral or lateral surface for the plurality of supports.

FIG. 18 is a perspective view of an expandable implant in which the means for linking comprises a stent, the means for linking being attached at a peripheral surface of the plurality of supports, the expandable implant being in an expanded condition.

FIG. 19 is a section view of an expandable implant in which the expandable implant comprises a first expandable implant and a second expandable implant, where the second expandable implant is insertable between the plurality of supports for the first expandable implant when the first expandable implant is in the expanded condition.

FIG. 20 is a partial section side view of an interbody implant system for use in a spine that includes a first vertebra and a second vertebra, the system comprising an expandable implant, the expandable implant comprising a plurality of supports and a central element that is insertable between the plurality of supports when the expandable implant is in the expanded condition.

FIG. 21 is a perspective view of the expandable implant of the system of FIG. 20 when the central element is inserted between the plurality of supports.

FIG. 22 is a perspective view of the central element of the system of FIG. 20.

FIG. 23 is a section view of a support in an expandable implant in which the means for linking is at least partially insertable within a hole or a groove in the plurality of supports.

FIG. 24 is a section view of two supports in an expandable implant in which the means for linking is at least partially insertable within a hole or a groove in the plurality of supports.

FIG. 25 is a partial section side view of an interbody implant system for use in a spine that includes a first vertebra and a second vertebra, the system comprising an expandable implant and means for expanding the expandable implant, in which the means for expanding comprises a plurality of wedges.

FIG. 26 is a partial section side view of the system of FIG. 25 during the inserting of the plurality of wedges between the plurality of supports, when the expandable implant is in a partially expanded condition.

FIG. 27 is a partial section side view of the system of FIG. 25, the view being taken prior to inserting the plurality of wedges between the plurality of supports.

FIG. 28 is a partial section side view of a means for expanding that comprises a plurality of wedges and a plurality of fins.

FIG. 29 is a section view of a means for expanding that comprises a plurality of wedges and a plurality of fins.

FIG. 30 is a partial section side view of an interbody implant system for use in a spine that includes a first vertebra and a second vertebra, the system comprising an expandable implant, the expandable implant comprising a plurality of supports and a central element that is insertable between the plurality of supports when the expandable implant is in the expanded condition.

FIG. 31 is a perspective view of the central element of the system of FIG. 30.

FIG. 32 is a partial section side view of an interbody implant system for use in a spine that includes a first vertebra and a second vertebra, the system comprising an expandable implant and a catheter for introducing bone graft material into the expandable implant when the expandable implant is in the expanded condition.

FIG. 33 is a side view of an expandable implant, for an interbody implant system, in which the means for linking comprises a spring.

FIG. 34 is a section view of the expandable implant of FIG. 33 when the expandable implant 21A is in an unexpanded condition.

FIG. 35 is a section view of the expandable implant of FIG. 33 when the expandable implant is in an expanded condition.

FIG. 36 is a section view of an expandable implant, for an interbody implant system, in which the expandable implant comprises a first expandable implant and a second expandable implant, where the second expandable implant is insertable between the plurality of supports for the first expandable implant when the first expandable implant is in the expanded condition.

FIG. 37 is a higher magnification view of a slideable connection between a outer spring and an inner spring in the expandable implant of FIGS. 33-36.

FIG. 38 is a side view of the expandable implant of FIG. 36.

FIG. 39 is a partial section side view of an interbody implant system for use in a spine that includes a first vertebra and a second vertebra, the system comprising an expandable implant and means for expanding the expandable implant, in which the means for expanding comprises a balloon and an inflation line that is connected to the balloon.

FIG. 40 is a partial section side view of the system of FIG. 33 when the balloon is inflated and the expandable implant is in the expanded condition.

FIG. 41 is a partial section side view of an interbody implant system comprising an expandable implant in which the plurality of supports includes a hole that extends from support first end to support second end.

FIG. 42 is a section view of the expandable implant of the system of FIG. 41.

FIG. 43 is a section view of an expandable implant, for an interbody implant system, in which the plurality of supports includes a groove and the means for linking is at least partially insertable into the groove.

FIG. 44 is a side view of a support, for an expandable implant, in which the support includes a ridge at the support first end or the support first end.

FIG. 45A is a section view of an expandable implant in which the means for linking comprises an elongate member.

FIG. 45B is a section view of the expandable implant of FIG. 45A.

FIG. 46 is a partial section side view of an interbody implant system in which for at least a majority of the plurality of supports the support height for a central region of the support is greater than the support height for a peripheral region of the support.

FIG. 47 is a partial section side view of an interbody implant system in which the support height differs among the plurality of supports.

FIG. 48 is a partial section side view of an interbody implant system in which the support height differs among the plurality of supports.

FIG. 49 is a partial section side view of a first vertebra and a second vertebra during the performance of a method that includes forming a channel, the channel having a channel diameter, a pedicle region, a central region, and an endplate region, wherein the channel diameter for the central region is greater than the channel diameter for the pedicle region and the channel diameter for the central region is greater than the channel diameter for the endplate region.

FIG. 50 is a partial section side view of a first vertebra and a second vertebra during the performance of a method that includes forming a channel, the channel having a channel diameter, a pedicle region, a central region, and an endplate region, wherein the channel diameter for the central region is greater than the channel diameter for the pedicle region and the channel diameter for the central region is greater than the channel diameter for the endplate region.

FIG. 51 is a partial section side view of a first vertebra and a second vertebra during the performance of a method that includes forming a channel, the channel having a channel diameter, a pedicle region, a central region, and an endplate region, wherein the channel diameter for the central region is greater than the channel diameter for the pedicle region and the channel diameter for the central region is greater than the channel diameter for the endplate region.

FIG. 52A is a section view, from the anterior, of a vertebral body in which a single channel is formed, the channel being located at an asymmetric position with respect to the sagittal plane.

FIG. 52B is a section view, from the anterior, of a vertebral body in which a single channel is formed, the channel being angled so that it intersects first endplate close to the sagittal plane.

FIG. 52C is an axial view of a vertebra (lumbar vertebra L5) in which a single channel is formed, with a single expandable implant installed in the vertebra at an asymmetric position with respect to the sagittal plane.

FIG. 53 is a partial section side view of two vertebrae and tools used in a method for treating a spine, during the forming of a curved channel in a first vertebra.

FIG. 54 is a partial section side view of two vertebrae and tools used in a method for treating a spine, during the forming of a curved channel in a first vertebra.

FIG. 55 is a partial section side view of two vertebrae and tools used in a method for treating a spine, during the forming of a curved channel in a first vertebra.

FIG. 56 is a partial section side view of two vertebrae and a channel formed in the caudal vertebra.

FIG. 57 depicts a steerable needle that may be used in forming a curved channel.

FIG. 58 depicts a steerable drilling tool that may be used in forming a curved channel.

### DETAILED DESCRIPTION OF THE INVENTION

Reference will now be made in detail to some embodiments, examples of which are illustrated in the accompanying drawings. In this description and in the appended claims, the terms ‘a’ or ‘an’ are used, as is common in patent documents, to include one or more than one. In this description and in the appended claims, the term ‘or’ is used to refer to a nonexclusive ‘or’, unless otherwise indicated.

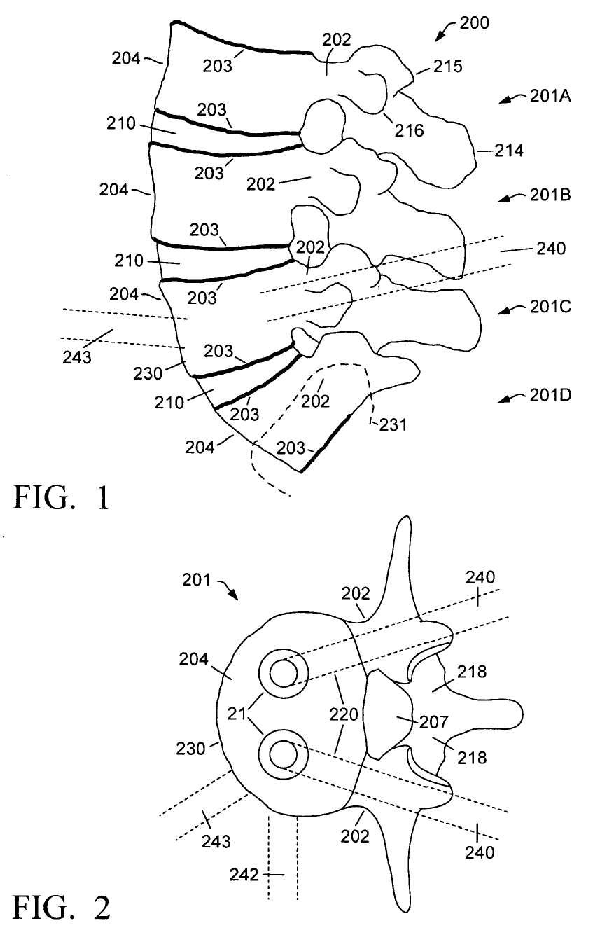

FIG. 1 is a side view of four vertebrae 201A, 201B, 201C and 201D, in the lumbar and sacral regions of a human spine 200. The depicted vertebrae 201A, 201B, 201C and 201D correspond to human vertebrae L3, L4, L5, and S1, respectively. FIG. 2 is an axial cephalad view of vertebra L4. Each vertebra 201 includes an anterior part, the body 204, and a posterior part, the vertebral arch, that consists of a pair of pedicles 202 and a pair of laminae 218. The body 204, the pedicles 202, and the laminae 218 together enclose an opening, the vertebral foramen 207; the spinal cord passes through the vertebral foramen 207. The first sacral (S1) vertebra 201D includes a portion of the auricular surface 231 of the sacrum.

The body 204 is composed of cancellous bone covered by a thin layer of cortical bone. Cortical bone is strong and compact, while cancellous bone is more cellular and has many apertures, so that it is less strong than cortical bone. Spinal discs (intervertebral discs) 210 located between the vertebral bodies 204 serve as shock absorbers that cushion the bodies 204. Each body 204 has two endplates 203, one on the superior (upper or cephalad) surface of the body 204, and one on the inferior (lower or caudal) surface of the body 204. A body wall 230 made of cortical bone extends between the superior and inferior endplates 203. Each endplate 203 includes a cortical bone layer and an external cartilage layer. The endplate 203 has a thickness of about one to several millimeters. Blood vessels in the cartilage layer supply nutrients to the adjacent spinal disc 210.

Surgical procedures for the spine 202 may employ various surgical approaches such as an anterior approach 243 or a posterior approach 240 or a lateral approach 242. These various surgical approaches are indicated by paired dashed lines in FIGS. 1 and 2. A transpedicular posterior approach 240 through a pedicle 202 is indicated in FIGS. 1 and 2. Embodiments described herein employ a channel 220 that extends through a vertebra 201 and an expandable implant 21 that is installed through the channel 220. The channel 220 may extend through a pedicle 202 and an endplate 203 of the vertebra 201, using a transpedicular approach 240, or the channel 220 may extend through the body wall 230 and through an endplate 203 of the vertebra 201, using an anterior approach 243 or a lateral approach 242. The embodiment depicted in FIG. 2, comprising two channels 220 and two expandable implants 21, employs a transpedicular posterior approach 240 to the channels 220.

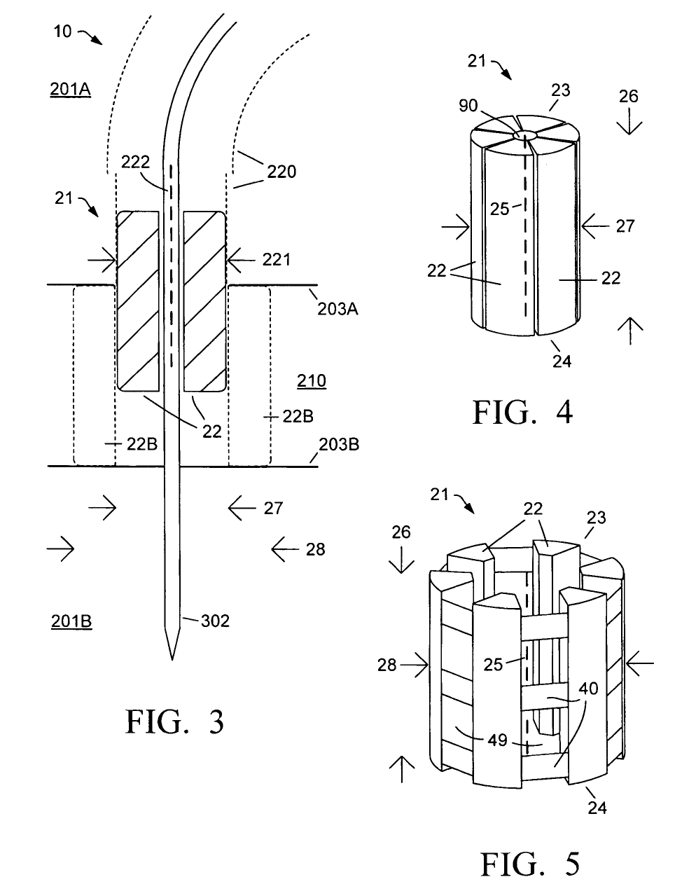

FIG. 3 is a partial section side view of an interbody implant system 10 for use in a spine that includes a first vertebra 201A and a second vertebra 201B, the system 10 comprising an expandable implant 21, the expandable implant 21 comprising a plurality of supports 22 and means 40 for linking the plurality of supports 22, in accordance with an embodiment. Means for linking 40 is indicated in FIG. 5. FIG. 4 is a perspective view of the expandable implant 21 of the system 10 of FIG. 3 when the expandable implant 21 is in an unexpanded condition. FIG. 5 is a perspective view of the expandable implant 21 of the system 10 of FIG. 3 when the expandable implant 21 is in an expanded condition. The first vertebra 201A has a first endplate 203A that is adjacent a spinal disc 210, and the second vertebra 201B has a second endplate 203B that is adjacent the spinal disc. The first vertebra 201A has a pedicle 202 and a body wall 230, as depicted in FIGS. 1, 2, and 6.

The expandable implant 21, when positioned between the endplates 203A and 203B and expanded to the expanded condition, may serve as an interbody implant or spacer that helps to stabilize and distract the vertebrae 201A and 201B. The expandable implant 21 may be used as an adjunct to a spinal fusion procedure, in which stabilization of the vertebrae 201 facilitates successful fusion of the vertebrae 201.

The plurality of supports 22 are capable of moving apart from one another whereby the expandable implant 21 is in an expanded condition, as depicted in FIG. 5. The dashed line silhouettes labelled 22B in FIG. 3 indicate the position of the plurality of supports 22 when the plurality of supports 22 have moved apart from one another. The plurality of supports 22 has a support first end 23 and a support second end 24 and a support axis 25 that extends from the support first end 23 to the support second end 24. The plurality of supports 22 has a support height 26. The expandable implant 21 has an unexpanded diameter 27 that is perpendicular to the support axis 25. The expandable implant 21 has an expanded diameter 28 when the expandable implant 21 is in the expanded condition, the expanded diameter 28 being perpendicular to the support axis 25.

In this description and in the appended claims, a statement that a diameter (expanded or unexpanded) is perpendicular to the support axis 25 means that the diameter, which is a scalar value, is measured in a plane that is perpendicular to the support axis 25. In this description and in the appended claims, the term “unexpanded diameter” means the largest dimension for the expandable implant 21 in any plane that is perpendicular to the support axis 25 when the expandable implant is in the unexpanded condition. In this description and in the appended claims, the term “expanded diameter” means the largest dimension for the expandable implant 21 in any plane that is perpendicular to the support axis 25 when the expandable implant is in the expanded condition. The expandable implant 21 may have a cross-sectional shape that is not circular. The diameter may vary between the support first end 23 and the support second end 24.

Each of the plurality of supports 22 is linked to at least another one of the plurality of supports 22 by the means for linking 40. In the embodiment of FIGS. 3-5, each of the plurality of supports 22 is linked to two other supports 22. In other embodiments, the plurality of supports 22 may comprise a smaller or larger number of supports 22 such as, for example, two or three or four or seven supports 22.

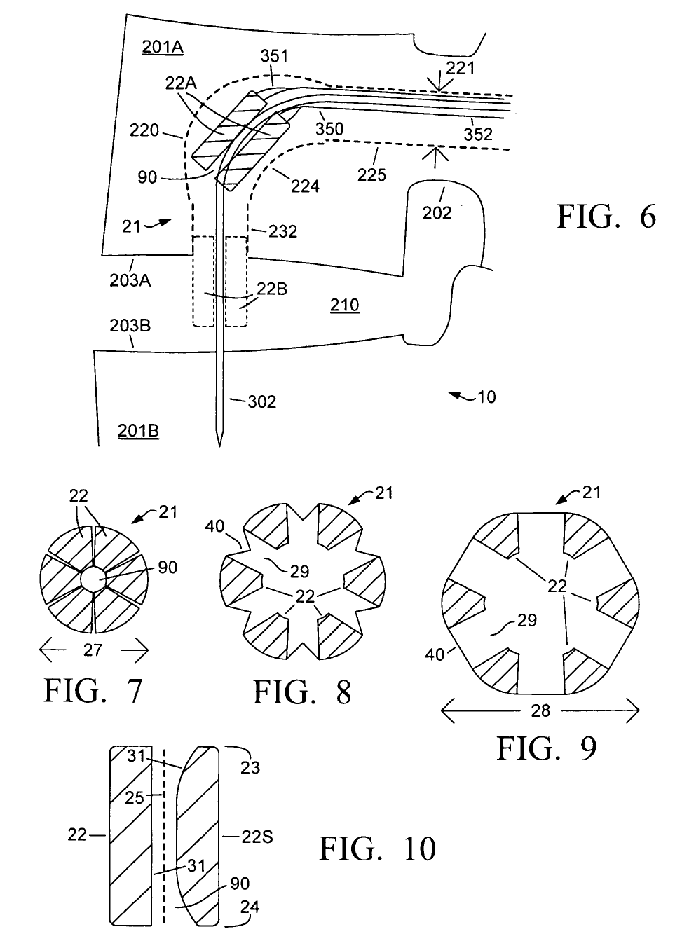

The unexpanded diameter 27 is configured to permit passage of the expandable implant 21 through a channel 220 in the first vertebra 201A. The channel 220 extends at least through the first endplate 203A, and the channel 220 extends through the pedicle 202 or the body wall 230, as described in connection with FIG. 2. FIG. 6 is a partial section side view of the system 10 of FIG. 3, the view being taken during passage of the expandable implant 21 through a channel 220 in the first vertebra 201A. In the embodiment of FIG. 6, the channel 220 extends through the pedicle 202, and the channel 220 has a pedicle region 225, a central region 224, and an endplate region 232. In another embodiment, the channel 220 may have a body wall region instead of a pedicle region 225.

In FIG. 6, the plurality of supports 22 is depicted at two different positions during passage of the expandable implant 21 through the channel 21. The plurality of supports 22A is depicted at a position within the central region 224 of channel 220. At a later stage, after advancing further through channel 220, the plurality of supports 22B is depicted at a position that is partly within the endplate region 232 of the channel and partly within the spinal disc 210. In the embodiment of FIGS. 3-6, the plurality of supports 22 includes a passage 90 for a guidewire 302. The expandable implant 21 may be advanced through channel 220 using a flexible driver 350 that includes a driver tip 351 and a flexible drive shaft 352 that includes a passage for the guidewire 302, or the expandable implant 21 may be advanced through channel 220 by other means, as described herein.

The expandable implant 21 is advanced through the channel 220 and into the spinal disc 210 between endplates 203A and 203B. The expandable implant 21 is then expanded to the expanded condition. The channel 220 has a channel axis 222 and a channel diameter 221. The channel axis 222 at the first endplate 203A is oblique or perpendicular to the first endplate 203A. The expanded diameter 28 is configured to be greater than the channel diameter 221 at the first endplate 203A. The dashed line silhouettes labelled 22B in FIG. 3 indicate the position of the plurality of supports 22 when the expandable implant 21 is in the expanded condition having the expanded diameter 28.

The support height 26 is configured to permit the support second end 24 to be positioned adjacent the second endplate 203B while the support first end 23 is positioned adjacent the first endplate 203A while the support axis 25 is oriented substantially perpendicular to the first endplate 203A. The dashed line silhouettes labelled 22B in FIG. 3 represent a plurality of supports 22 in which the support second end 24 is positioned adjacent the second endplate 203B while the support first end 23 is positioned adjacent the first endplate 203A while the support axis 25 is oriented substantially perpendicular to the first endplate.

Much of the information described in connection with FIGS. 2-6 applies generally to other embodiments. Thus, this general information is not repeated in the description of each embodiment. It is understood that every system 10 embodiment comprises an expandable implant 21 that comprises a plurality of supports 22 and a means for linking 40, and having the features described in the previous paragraphs in connection with the embodiment of FIGS. 3-6.

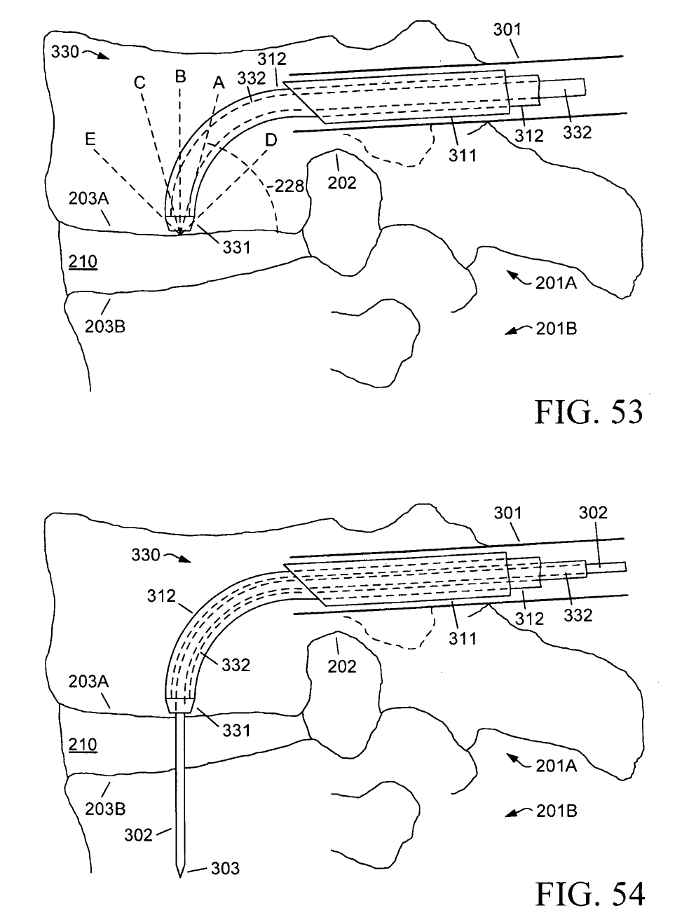

The phrases “substantially perpendicular” and “oblique or perpendicular” each indicate a range for an angle 228 relative to the first endplate 203A. As used herein and in the appended claims, the phrase “substantially perpendicular” means an angle 228 having a value that is greater than or equal to 75 degrees and less than or equal to 105 degrees, as depicted in FIG. 53. A “substantially perpendicular” angle 228 for the support axis 25 is an angle 228 having any value, either integral or non-integral, between 75 degrees and 105 degrees. FIG. 53 includes three dashed lines labeled A, B, and C that intersect first endplate 203A at angles 228 having values of 75 degrees, 90 degrees, and 105 degrees, respectively.

As used herein and in the appended claims, the phrase “oblique or perpendicular” means an angle 228 having a value that is greater than or equal to 45 degrees and less than or equal to 135 degrees, as depicted in FIG. 53. An “oblique or perpendicular” angle 228 for the channel axis 222 is an angle 228 having any value, either integral or non-integral, between 45 degrees and 135 degrees. FIG. 53 includes two dashed lines labelled D and E that intersect first endplate 203A at angles 228 having values of 45 degrees and 135 degrees, respectively.

In an embodiment such as that of FIG. 6, in which the channel 220 extends through the pedicle 202 for the first vertebra 201A, the unexpanded diameter 27 may be further configured to permit passage of the expandable implant 21 through the pedicle region 225 for the channel. In another embodiment, the channel 220 may extend through the body wall 230 rather than the pedicle 202. The pedicle width 206 and pedicle height 205 may guide the selection of the channel diameter 221 for the pedicle region 225. Dimensions of vertebrae are discussed in connection with Table 2.

As used herein and in the appended claims, the term “spinal disc” means a normal spinal disc that is not injured or diseased and that has not been manipulated surgically and also means a spinal disc that has been injured or diseased or manipulated surgically so that some or all of the tissue between the first endplate 203A and the second endplate 203B has been removed or altered.

FIGS. 7-9 depict the expandable implant 21 of the system 10 of FIG. 3 during the transition from the unexpanded condition to the expanded condition. FIG. 7 is a section view of the expandable implant 21 of the system 10 of FIG. 3 when the expandable implant 21 is in an unexpanded condition. In FIGS. 7-9, the plane of section is perpendicular to support axis 25. FIG. 8 is a section view of the expandable implant 21 of the system 10 of FIG. 3 when the expandable implant 21 is in a partially expanded condition. FIG. 9 is a section view of the expandable implant 21 of the system 10 of FIG. 3 when the expandable implant 21 is in an expanded condition.

As used herein and in the appended claims, the term “expanded condition” means a partially expanded condition as in FIG. 8 or a fully expanded condition as in FIG. 9. In the embodiment of FIGS. 3-9, the plurality of supports 22 are close together in the unexpanded condition, with very small spaces between lateral surfaces 33. In another embodiment, there may be larger spaces or gaps 29 between the plurality of supports 22 in the unexpanded condition. When the expandable implant 21 is in the partially expanded condition as in FIG. 8, there are gaps 29 between the plurality of supports 22. The gaps 29 become larger as the expandable implant 21 attains the expanded condition that is depicted in FIGS. 5 and 9.

In the embodiment of FIGS. 3-9, the means for linking 40 is a sheet 42 with plural openings 49 in the means for linking 40, as shown in FIG. 5. The means for linking 40 for the FIGS. 3-9 embodiment may alternatively be described as a plurality of means for linking 40, with three means for linking 40 linking each pair of supports 22, c.f. FIG. 5. In the embodiment of FIGS. 3-9, the means for linking 40 may be folded into the gaps 29 when the expandable implant 21 is in the unexpanded condition, c.f. FIG. 8. The means for linking 40 unfolds progressively as the plurality of supports 22 move apart from one another, as shown in FIG. 9.

In other embodiments, means for linking 40 may take many different forms. In every embodiment, each of the plurality of supports 22 is linked to at least another one of the plurality of supports 22 by the means for linking 40. In one embodiment, means for linking 40 may be, for example, an elongate member formed into a ring or polygon that simply surrounds the plurality of supports 22, so that the plurality of supports 22 are capable of moving apart from one another to the expanded condition, but with the means for linking 40 retaining all of the plurality of supports 22 within the surrounding means for linking 40. In this description and in the appended claims, a plurality of supports 22 that is surrounded by a means for linking 40 is one embodiment of a plurality of supports 22 in which each of the plurality of supports 22 is linked to at least another one of the plurality of supports 22 by the means for linking 40. In an expandable implant 21 in which the plurality of supports 22 comprises two supports 22, as in the embodiments of FIGS. 8-9 and 33-35, each of the plurality of supports 22 is linked to another one of the plurality of supports 22 by the means for linking 40.

In the embodiment of FIGS. 3-9, expandable implant 21 includes a passage 90 for a guidewire 302. In another embodiment, guidewire 302 and passage 90 may be omitted and expandable implant 21 may be advanced through channel 220 using a steerable driver tool that does not rely upon a guidewire 302. A steerable driver tool may employ a steering mechanism such as a set of telescoping tubes or a tension wire, as described in connection with FIGS. 53, 57, and 58.

Guidewire 302 is curved where it passes through the central region 224 of channel 220, as shown for example in FIG. 6. If passage 90 is straight and relatively narrow, the curved portion of guidewire 302 may not fit easily within passage 90, as indicated by the overlap of guidewire 302 and plurality of supports 22A that is depicted in FIG. 6. Guidewire 302 may be bent sharply at support first end 23 or at support second end 24, which may cause guidewire 302 to bind so that expandable implant 21 cannot advance. To accommodate the curvature of guidewire 302 and to reduce binding, expandable implant 21 may include a passage 90 that varies in width between the support first end 23 and the support second end 24. For example, passage 90 may have an hourglass shape that is wide at both ends and narrow in the middle. In another example, passage 90 may have a vase shape that is narrow at a first end and progressively wider towards the second end.

FIG. 10 is a section view of an expandable implant 21 in which a passage 90 for a guidewire 302 is partially curved, in accordance with an embodiment. In the section view of FIG. 10, the plane of section is parallel to support axis 25. In the embodiment of FIG. 10, the expandable implant 21 includes a passage 90 for a guidewire 302, the passage 90 extending from the support first end 23 to the support second end 24, and at least one of the plurality of supports 22 has a central surface 31 that is at least partially curved relative to the support axis 25, the at least partially curved central surface 31 defining at least a portion of the passage 90 for the guidewire 302. The support 22 that is at the left side has a central surface 31 that is straight. The support 22S that is at the right side has a central surface 31 that is at least partially curved relative to the support axis 25, resulting in a widening of passage 90 at support first end 23 and at support second end 24.

In the embodiment of FIG. 10, it is possible that only one or a few of the plurality of supports 22 has a curved central surface 31, in order to minimize the reduction in first end surface area and second end surface area that results from widening of passage 90. In the FIG. 10 embodiment, the partially curved passage 90 is an irregularly shaped slot that extends into one or a few of the plurality of supports 22.

In another embodiment, the passage 90 for a guidewire 302 may be offset from the support axis 25. For example, one of the supports 22 could be much narrower than the other supports 22, or there could be a gap 29 between a pair of supports 22 in the unexpanded condition, with the passage 90 being within the gap 29.

In another embodiment, guidewire 302 may have varied flexibility for individual portions of guidewire 302 in order to reduce binding. For example, a first flexibility for a central portion of the guidewire 302 may be greater than a second flexibility for a distal portion of the guidewire 302, the distal portion being capable of being positioned at least partially within second vertebra 201B or spinal disc 210. The greater flexibility for the central portion of guidewire 302 may facilitate sliding of expandable implant 21 along guidewire 302 when guidewire 302 is curved or bent, as in central region 224 of channel 220. The lesser flexibility for the distal portion of guidewire 302 may facilitate positioning of the distal portion and may also facilitate aligning of expandable implant 21 relative to first endplate 203A and second endplate 203B (FIG. 6).

In the embodiment of FIGS. 3-9, the lumen or passage 90 has a lumen diameter that is relatively small compared to the unexpanded diameter 27 for expandable implant 21 (FIG. 7). In other embodiments, expandable implant 21 may have a lumen or passage 90 with a lumen diameter that is larger relative to the unexpanded diameter 27, or expandable implant 21 may have a cavity or space between the plurality of supports 22 at the support first end 23 or at the support second end 24 or at an intermediate position. If a lumen diameter or cavity diameter is large, this reduces the first end surface area or the second end surface area, compared to an expandable implant 21 with a small lumen diameter or cavity diameter.

As used herein and in the appended claims, the term “first end surface area” means the sum of the areas for the individual supports 22 at the support first end 23, and the term “second end surface area” means the sum of the areas for the individual supports 22 at the support second end 24. As used here and in the appended claims, the term “first end envelope area” means the overall area for support first end 23 in the unexpanded condition without subtracting the area of any gap 29 or lumen or passage 90 or cavity. As used here and in the appended claims, the term “second end envelope area” means the overall area for support second end 24 in the unexpanded condition without subtracting the area of any gap 29 or lumen or passage 90 or cavity.

A large first end surface area or a large second end surface area may help expandable implant 21 to stabilize and distract the vertebrae 201, and may help to reduce subsidence of expandable implant 21 into the endplates 203. Thus, it may be advantageous for an expandable implant 21 to have a first end surface area that is large relative to the first end envelope area and also to have a second end surface area that is large relative to the second end envelope area. For example, the first end surface area may be greater than 50 percent of the first end envelope area, or greater than a higher percent such as 60 or 70 or 80 or 90 percent.

In the embodiment of FIGS. 3-9, the expandable implant 21 may have, for example, an unexpanded diameter 27 of 6.5 millimeters and a lumen diameter of 1.5 millimeters, so that the first end surface area is greater than or equal to 90 percent of the first end envelope area and the second end surface area is greater than or equal to 90 percent of the second end envelope area; the actual percent is about 94 percent for the FIG. 3-9 embodiment. In another example, an expandable implant 21 may have an unexpanded diameter 27 of 7.1 millimeters and a lumen diameter of 5.0 millimeters and minimal space between lateral surfaces 33, corresponding to a first end surface area that is greater than or equal to about 50 percent of the first end envelope area. In another example, an expandable implant 21 may have an unexpanded diameter 27 of 8.0 millimeters and a lumen diameter of 4.3 millimeters and minimal space between lateral surfaces 33, corresponding to a first end surface area that is greater than or equal to about 70 percent of the first end envelope area.

For an expandable implant 21 in a system 10, a ratio of the expanded diameter 28 to the unexpanded diameter 27 may be greater than or equal to 1.75. In one embodiment, the expandable implant 21 may have an unexpanded diameter 27 of 6.5 millimeters and a lumen diameter of 1.5 millimeters and an expanded diameter 28 of 11.5 millimeters. In such an embodiment, a ratio of the expanded diameter 28 (11.5 millimeters) to the unexpanded diameter 27 (6.5 millimeters) is greater than or equal 1.75.

In the embodiment of FIG. 6, the channel 220 has a variable diameter. The channel diameter 221 for the central region 224 is greater than the channel diameter 221 for the pedicle region 225 of the channel 220 and greater than the channel diameter for the endplate region 232 of the channel 220. The variation in channel diameter 221 serves to accommodate the different constraints upon channel diameter 221 for the pedicle region 225, the central region 224, and the endplate region 232.

In general, a large channel diameter 221 would allow an expandable implant 21 to have an unexpanded diameter 27 that is large and yet still permit passage of the expandable implant 21 through the channel 220. When a channel 220 is curved, as in the central region 224 in the FIG. 6 embodiment, any element, such as expandable implant 21, may become stuck in the curved region and unable to advance. To prevent the element becoming stuck, the element may be made shorter or narrower or the element may be tapered at one or both ends, or the channel diameter 221 may be made somewhat larger than the element diameter.

A shortened expandable implant 21 having a reduced height 26 may not be tall enough to serve as an interbody spacer, unless it is stacked. A narrow or tapered expandable implant 21 with an unexpanded diameter 27 that is small may have an expanded diameter 28 that is small, which would undermine the spacer function of the expandable implant 21. In other words, the expanded spacer “footprint” would be small. In addition, narrowing or tapering of expandable implant 21 would reduce the first end surface area or the second end surface area, which might encourage subsidence into the vertebrae 201, thus undermining the spacer function of the expandable implant 21. Thus, for a curved central region 224 of a channel 220, it may be useful to make the channel diameter 221 somewhat larger than the unexpanded diameter 27 of the expandable implant 21.

In the pedicle region 225, however, a smaller channel diameter 221 may be advantageous in order to maintain the strength of the pedicle 202. A smaller channel diameter 221 may be advantageous in the endplate region 232 as well, because a smaller channel diameter 221 preserves more of the first endplate 203A and thus helps to maintain the strength of the vertebral body 204. The foregoing considerations lead to the channel 220 embodiment depicted in FIG. 6: a channel 220 with a larger channel diameter 221 for the central region 224 and a smaller channel diameter 221 for the pedicle region 225 or the endplate region 232.

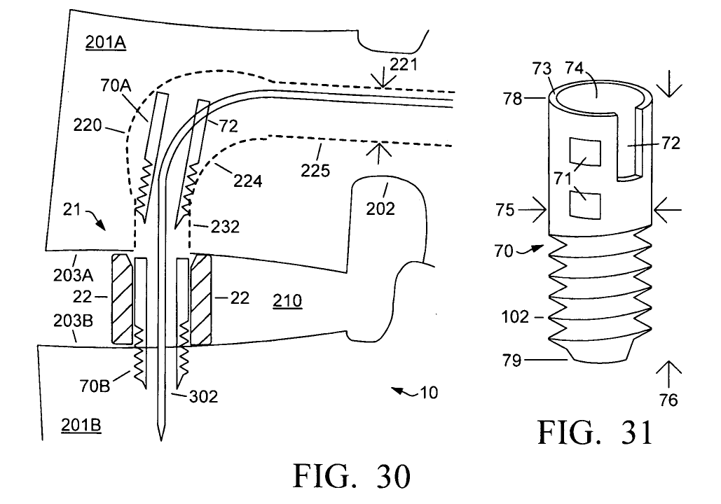

FIG. 30 depicts an embodiment that employs a channel 220 similar to that of FIG. 6, with a central region 224 having a channel diameter 221 that is greater than the channel diameter 221 for the pedicle region 225 or the endplate region 232. The expandable implant 21 of FIG. 30 includes a central element 70 that is fairly long, and the central element 70 may need to be aligned properly relative to the first endplate 203A and the plurality of supports 22. The large channel diameter 221 in the central region 224 may facilitate aligning the central element 70 relative to the first endplate 203A and the plurality of supports 22.

In another embodiment, a channel 220 may be angled upward in pedicle region 225, as described in connection with FIG. 27.

In the embodiments of FIGS. 6 and 30, the channel diameter 221 for the central region 224 is greater than the channel diameter 221 for the pedicle region 225 and the channel diameter 221 for the central region 224 is greater than the channel diameter 221 for the endplate region 232. In another variable diameter embodiment, the channel diameter 221 for the central region 224 may be greater than the channel diameter 221 for the pedicle region 225 but may be equal to or less than the channel diameter for the endplate region 232. In another variable diameter embodiment, the channel diameter 221 for the central region 224 may be greater than the channel diameter 221 for the endplate region 232 but may be equal to or less than the channel diameter 221 for the pedicle region 225.

In another variable diameter embodiment, a channel 220 may extend through the body wall 230 and through the first endplate 203A, the channel 220 having a channel diameter 221 for a central region 224 that is greater than the channel diameter 221 at the body wall 230 or the endplate region 232.

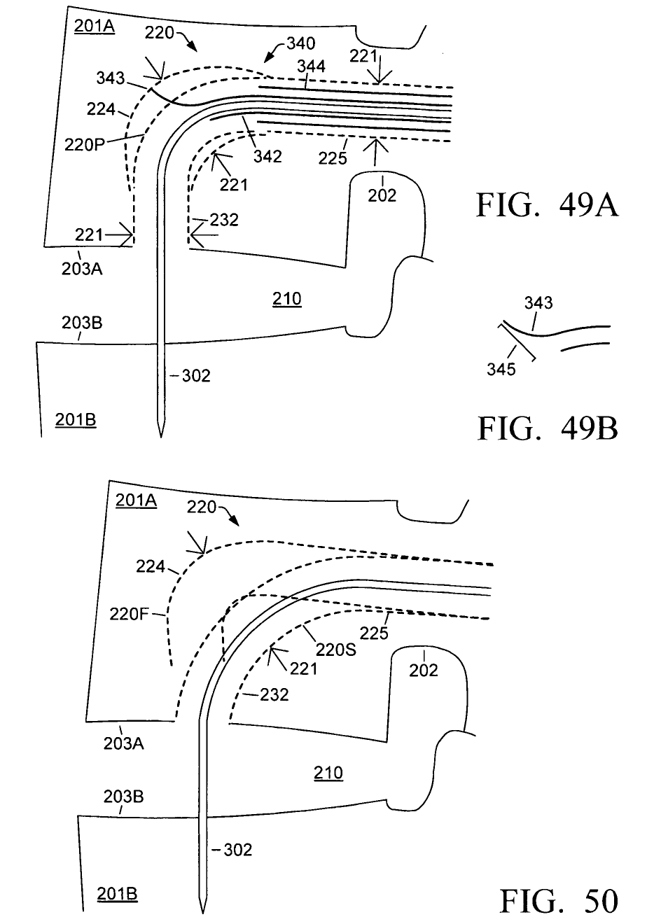

A method of forming a channel 220 with a large channel diameter 221 in the central region 224 is described in connection with FIGS. 49-51.

FIG. 11 is a partial section side view of an interbody implant system 10 for use in a spine that includes a first vertebra 201A and a second vertebra 201B, the system 10 comprising an expandable implant 21 and means 50 for expanding the expandable implant 21, in which the means for expanding 50 comprises a wedge 51, in accordance with an embodiment. In the FIG. 11 embodiment, the leading end of the wedge 51 is rounded. FIG. 12 is a partial section side view of an interbody implant system 10 for use in a spine that includes a first vertebra 201A and a second vertebra 201B, the system 10 comprising an expandable implant 21 and means 50 for expanding the expandable implant 21, in which the means for expanding 50 comprises a wedge 51, in accordance with an embodiment. In the FIG. 12 embodiment, the wedge 51 has a leading end with a profile that is triangular.

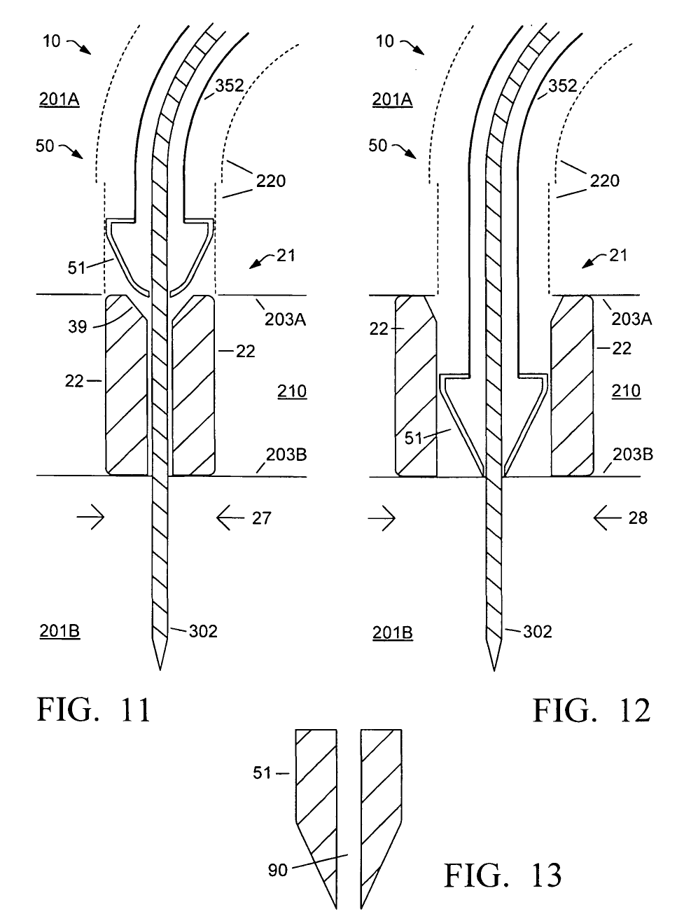

FIG. 13 is a section view of a means for expanding 50 that comprises a wedge 51, in accordance with an embodiment. In the section view of FIG. 13, the plane of section is parallel to support axis 25. The wedge 51 of the FIG. 13 embodiment is mainly solid, with a narrow passage 90 for a guidewire 302, in contrast to the FIG. 11 and FIG. 12 embodiments in which the wedge 51 has a thin wall enclosing a large lumen.

Wedge 51 is insertable between the plurality of supports 22. Wedge 51 is capable of exerting force upon the plurality of supports 22 so that the plurality of supports 22 move apart and the expandable implant 21 attains an expanded condition. In the FIG. 11 embodiment, wedge 51 is depicted at a time before inserting of wedge 51 between the plurality of supports 22, with the expandable implant 21 in an unexpanded condition. In the FIG. 12 embodiment, wedge 51 is depicted at a time after inserting of wedge 51 between the plurality of supports 22, when wedge 51 has advanced to contact second endplate 203B, with the expandable implant 21 in an expanded condition. The plurality of supports 22 may include a notch 39 at support first end 23, to facilitate insertion of wedge 51 between the plurality of supports 22.

In another embodiment, wedge 51 may include a fin 52 that is insertable between the plurality of supports 22. FIGS. 28 and 29 depict an embodiment in which a wedge 51 comprises a plurality of wedges 51A-C and a plurality of fins 52. In any embodiment that employs a wedge 51 as the means for expanding 50, a fin 52 on wedge 51 may help to prevent rotation or twisting of the plurality of supports 22 while the supports 22 are forced to move apart by the wedge 51.

Wedge 51 may be attached to flexible drive shaft 352, as in the FIGS. 11 and 12 embodiments, enabling withdrawal of wedge 51 from expandable implant 21 after expanding is complete. In one embodiment, the expandable implant 21 in the FIGS. 11 and 12 embodiments may be advanced through channel 220 by a separate flexible driver 350 which is withdrawn prior to advancing of means for expanding 50. In another embodiment, expandable implant 21 and means for expanding 50 may be advanced together through channel 220, with a driver tip 351 pressing against expandable implant 21. For example, driver tip 351 may be a blade or short cylinder that presses against expandable implant 21 for advancing through channel 220, the driver tip later retracting through a slot in wedge 51 so that wedge 51 may be inserted between the plurality of supports 22.

In another embodiment, wedge 51 may be advanced by a flexible driver 350 with a driver tip 351 that presses against wedge 51, similar to the flexible driver 350 depicted in FIG. 6, with the wedge 51 not attached to the flexible drive shaft 352. In such an embodiment, wedge 51 may remain inserted between the plurality of supports 22 after expanding is complete. In such an embodiment, wedge 51 serves two roles: wedge 51 serves as a means for expanding 50, and wedge 51 also serves as a central element 70 that helps to stabilize the plurality of supports 22, similar to the central elements 70 described in connection with FIGS. 20 and 30.

In another embodiment, wedge 51 may comprise bone or bone graft substitute. In such an embodiment, wedge 51 may be mainly or entirely solid, as in the FIG. 13 embodiment, so that wedge 51 will be strong for exerting force upon the plurality of supports 22. In such an embodiment, wedge 51 serves three roles: wedge 51 serves as a means for expanding 50, and wedge 51 also serves as a central element 70, and wedge 51 serves as a bone growth substrate that assists fusion of vertebrae 201.

As used herein and in the appended claims, the term “bone” means autograft or allograft bone. As used herein and in the appended claims, the term “bone graft substitute” means any material that is used as a substrate that is intended to promote formation of live bone. For example, bone graft substitute may include materials such as hydroxyapatite or synthetic materials and may include bone growth promoting agents such as bone morphogenetic protein (BMP).

In the embodiments of FIGS. 11-13, wedge 51 includes a lumen or passage 90 for a guidewire 302. In another embodiment, passage 90 may be omitted and wedge 51 may be advanced through channel 220 using a steerable driver tool that does not rely upon a guidewire 302. A steerable driver tool may employ a steering mechanism such as a set of telescoping tubes or a tension wire, as described in connection with FIGS. 53, 57, and 58.

In the embodiments of FIGS. 4-9, 14-19 and 21-24, means for linking 40 (or 42) help to stabilize the plurality of supports 22. Means for linking 40 may take many different forms. Means for linking 40 may comprise a plurality of means for linking 40. Means for linking 40 may comprise any type of elongate member such as a string 43 or a rod 44 or a wire 41. Means for linking 40 may comprise a sheet 42, with or without openings 49, or a mesh or stent 45. Means for linking 40 may comprise a spring 48 such as a flexible arc or a helical coil. Means for linking 40 may comprise a hinge 38. Means for linking 40 may be at least partially insertable within a hole 34 (FIG. 20, 23) or a groove 35 in the plurality of supports 22 (FIG. 24). Means for linking 40 may comprise a separate piece that is joined to the plurality of supports 22 using, for example, an adhesive or by embedding a portion of the piece within the plurality of supports 22 using a polymer molding process.

Means for linking 40 may comprise an extension of the supports 22, rather than a separate piece, the extension of the supports 22 being formed by, for example, a machining process or a polymer molding process. In this description and in the appended claims, when it is stated that a “means for linking is attached at” a surface or an end of a plurality of supports 22, this statement encompasses a means for linking 40 that is a separate piece and also a means for linking 40 that is an extension 37 of the plurality of supports 22.

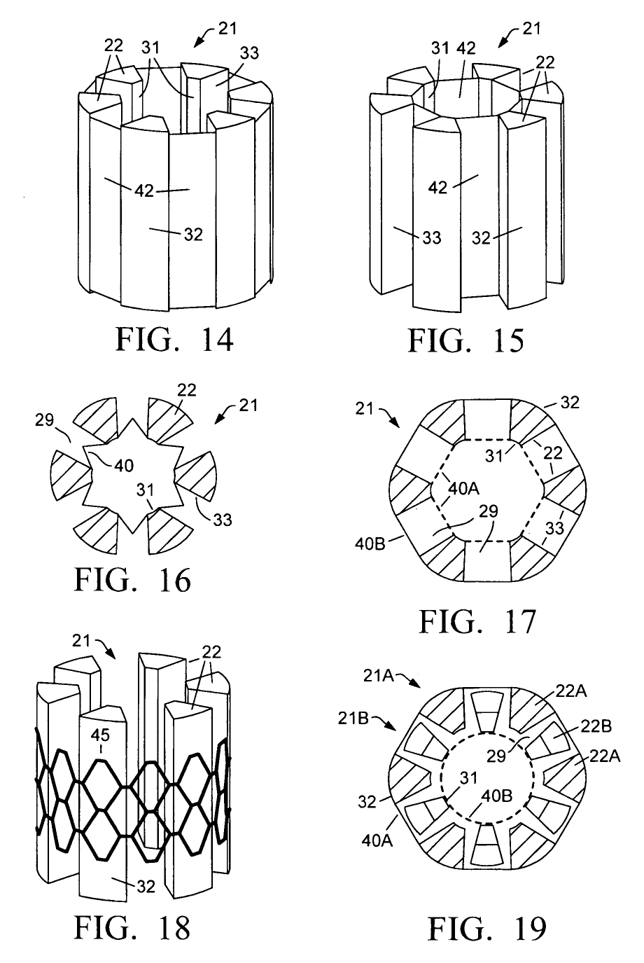

FIG. 14 is a perspective view of an expandable implant 21 in which the means for linking 40 comprises a sheet 42, the means for linking (sheet 42) being attached at a peripheral surface 32 (FIG. 14) or a lateral surface 33 (FIG. 15) of the plurality of supports 22. The means for linking 40 (sheet 42) in the FIG. 14 embodiment is similar to the means for linking 40 in the FIG. 5 embodiment, but the sheet 42 in the FIG. 14 embodiment does not include openings 49, as in the FIG. 5 embodiment. In any embodiment that comprises a sheet 40 or 42, a mesh 45 may be substituted for the sheet 42.

Sheet 42 may comprise a plurality of sheets 42, as depicted in FIG. 14. In another embodiment, sheet 42 may comprise a continuous sheet 42 that surrounds the plurality of supports 22. In such an embodiment, the continuous sheet 42 may simply surround the plurality of supports 22 without being attached using an adhesive or other means, although attachment may confer greater stability.

An embodiment similar to the FIG. 14 embodiment may be formed as an integral piece using a polymer molding process. In such an embodiment, the means for linking 40 (sheets 42) may be formed as thin flexible regions within the integral piece, the thin flexible regions being extensions of the plurality of supports 22 within the integral piece.

FIG. 15 is a perspective view of an expandable implant 21 in which the means for linking 40 comprises a sheet 42, the means for linking 40 (sheet 42) being attached at a central surface 31 or a lateral surface 33 of the plurality of supports 22, the expandable implant 21 being in an expanded condition, in accordance with an embodiment. The FIG. 15 embodiment is similar to the FIG. 14 embodiment, except for the positioning and attachment of the means for linking 40 (sheet 42). The sheet 42 may comprise a plurality of sheets 42 or a continuous sheet 42.

FIG. 16 is a section view of an expandable implant 21, the expandable implant 21 being in a partially expanded condition, in which the means for linking 40 is attached at a central surface 31 or a lateral surface 33 of the plurality of supports 22. In the section view of FIG. 16, the plane of section is perpendicular to support axis 25 (c.f. FIG. 4,5). In the embodiment of FIG. 16, the means for linking 40 is folded into the gaps 29 when the expandable implant 21 is in the unexpanded or partially expanded condition. The means for linking 40 unfolds progressively as the plurality of supports 22 move apart from one another. In the embodiment of FIG. 16, means for linking 40 may comprise a sheet 42 or a mesh 45 or a string 43 or a wire or any other flexible elongate member.

In another embodiment, means for linking 40 may be attached at the support first end 23 or the support second end 24 for the plurality of supports 22. In such an embodiment, means for linking 40 may comprise a sheet 42 or a mesh 45 or any type of flexible elongate member 41 that is folded into the gaps 29 between the plurality of supports 22 when the expandable implant 21 is in the unexpanded or partially expanded condition.

FIG. 17 is a section view of an expandable implant 21 that includes a first means for linking 40A that is attached at a central surface 31 or a lateral surface 33 for the plurality of supports 22 and a second means for linking 40B that is attached at a peripheral surface 32 or a lateral surface 33 for the plurality of supports 22, in accordance with an embodiment. In the section view of FIG. 17, the plane of section is perpendicular to support axis 25. In the embodiment of FIG. 17, means for linking 40A and 40B may comprise a sheet 42 or a mesh or stent 45 or any type of flexible elongate member, and means for linking 40A and 40B may comprise different types.

FIG. 18 is a perspective view of an expandable implant 21 in which the means for linking comprises a stent or mesh 45, the means for linking 45 being attached at a peripheral surface 32 of the plurality of supports 22, the expandable implant 21 being in an expanded condition, in accordance with an embodiment. In another embodiment, the stent 45 may be attached at a central surface 31 of the plurality of supports 22.

In some embodiments of the invention, as shown in FIG. 3 or FIG. 20, while expandable implant 21 is advancing through channel 220, it remains in the unexpanded condition. Various means may be used to prevent moving apart of the plurality of supports 22 during the advancing through channel 220. If the means for linking 40 is a stent 45, as in the FIG. 18 embodiment, the stent 45 may serve to hold together the plurality of supports 22 during the advancing through the channel 220. In another embodiment, the plurality of supports 22 may be encircled by a band or membrane or filament that is easily severable at one or more positions, so that the band or membrane or filament stays intact within channel 220 and then breaks when the expandable implant 21 begins expanding within spinal disc 210.

In another embodiment, a flexible driver 350, as shown for example in FIG. 6, may comprise a retractable sleeve at the distal (leading) end of the flexible driver 350, with the sleeve serving to hold together the plurality of supports 22 during the advancing through the channel 220, the sleeve being retracted after the expandable implant 21 arrives at the spinal disc 210. In another embodiment, the retractable sleeve may be replaced by a plurality of retractable prongs, each prong engaging one of the plurality of supports 22.

FIG. 19 is a section view of an expandable implant 21 in which the expandable implant 21 comprises a first expandable implant 21A and a second expandable implant 21B, where the second expandable implant 21B is insertable between the plurality of supports 22A for the first expandable implant 21A when the first expandable implant 21A is in the expanded condition, in accordance with an embodiment. In the section view of FIG. 19, the plane of section is perpendicular to support axis 25. First expandable implant 21A comprises a first means for linking 40A that is attached at a peripheral surface 32 of the plurality of supports 22A. Second expandable implant 21B comprises a second means for linking 40B that is attached at a central surface 31 of the plurality of supports 22B.

In an embodiment comprising two expandable implants 21A and 21B, such as that of FIG. 19, the combined first end surface area is the sum of the first end surface area for expandable implant 21A and the first end surface area for expandable implant 21B. The combined first end surface area is greater than the first end surface area for an expandable implant 21 that comprises a single expandable implant 21. Similarly, the combined second end surface area is greater than the second end surface area for a single expandable implant 21. The greater first end surface area and second end surface area may help the expandable implant 21 to stabilize and distract the vertebrae 201, and may help to prevent subsidence of expandable implant 21 into endplates 203.

In the embodiment of FIG. 19, at least a majority of the plurality of supports 22B for the second expandable implant 21B are dimensioned to be insertable into a plurality of gaps 29 between the plurality of supports 22A for the first expandable implant 21A in the expanded condition. The embodiment of FIG. 19 is installed in several steps. Initially, the first expandable implant 21A is advanced through channel 220 to the spinal disc 210 and expanded to the expanded condition. Next, the second expandable implant 21B is advanced through channel 220 and inserted between the plurality of supports 22A for the first expandable implant 21A within the space that is created by the moving apart of the plurality of supports 22A. Finally, the second expandable implant 21B is expanded so that the plurality of supports 22B move at least partially into the gaps 29 between the plurality of supports 22A.

The embodiment of FIGS. 36 and 38 is another example of an expandable implant 21 that comprises a first expandable implant 21A and a second expandable implant 21B, where the second expandable implant 21B is insertable between the plurality of supports 22A for the first expandable implant 21A when the first expandable implant 21A is in the expanded condition.

In another embodiment, the second expandable implant 21B may be capable of pressing outward on the first expandable implant 21A for causing a further moving apart of the plurality of supports 22A for the first expandable implant 21A, wherein the further moving apart causes an increase in the expanded diameter 28 for the first expandable implant 21A. In such an embodiment, the second expandable implant 21B serves as an additional means for expanding 50. This embodiment is installed in several steps. Initially, the first expandable implant 21A is advanced through channel 220 to the spinal disc 210 and expanded to the expanded condition. Next, the second expandable implant 21B is advanced through channel 220 and inserted between the plurality of supports 22A for the first expandable implant 21A within the space that is created by the moving apart of the plurality of supports 22A. Finally, the second expandable implant 21B is expanded so that the plurality of supports 22B presses outward on the first expandable implant 21A for causing a further moving apart of the plurality of supports 22A for the first expandable implant 21A. In such an embodiment, the means for linking 40 for the first expandable implant 21A is made large enough to accommodate the further moving apart.

FIG. 20 is a partial section side view of an interbody implant system 10 for use in a spine that includes a first vertebra 201A and a second vertebra 201B, the system 10 comprising an expandable implant 21, the expandable implant 21 comprising a plurality of supports 22 and a central element 70 that is insertable between the plurality of supports 22 when the expandable implant 21 is in the expanded condition, in accordance with an embodiment. In the FIG. 20 embodiment, the central element 70 has a central element diameter 75 that is configured to permit passage of the central element 70 through the channel 220.

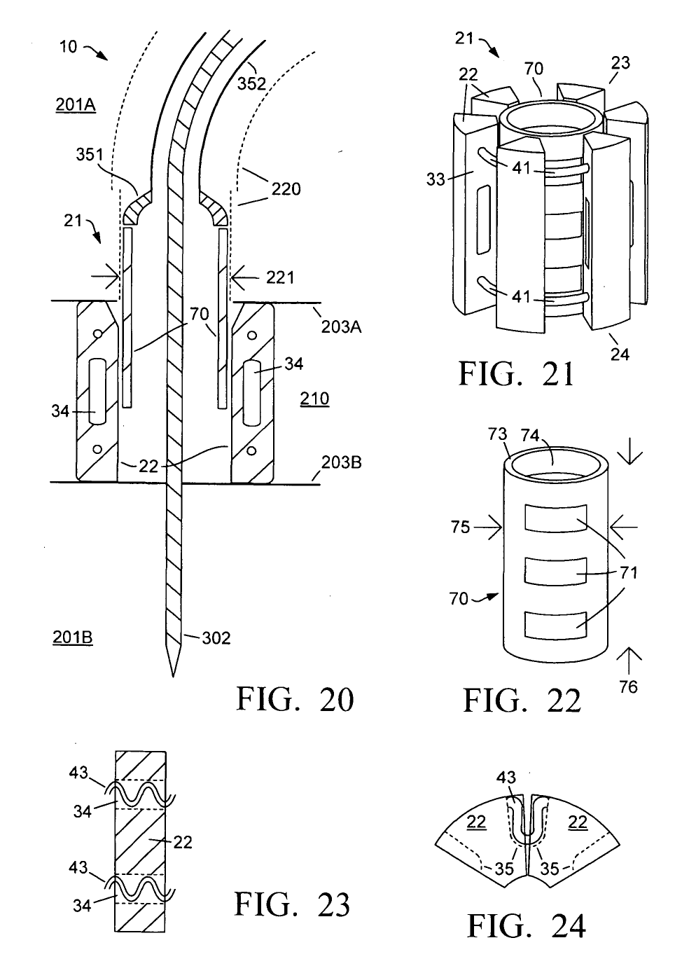

The central element 70 helps to stabilize the plurality of supports 22. FIG. 21 is a perspective view of the expandable implant 21 of the system 10 of FIG. 20 when the central element 70 is inserted between the plurality of supports 22. In the FIG. 20-21 embodiment, means for linking 40 is an elongate member 41, such as a string 43, that is attached at a lateral surface 33 of the plurality of supports 22 or that is partially embedded within the plurality of supports 22. Elongate member 41 is discussed further in connection with FIGS. 23 and 24.

In the view of FIG. 20, expandable implant 21 is depicted in the expanded condition, with central element 70 having advanced to the endplate region of channel 220 and central element 70 inserted between the plurality of supports 22. Central element 70 is advanced through channel 220 by a flexible driver 350 (see FIG. 6) having a driver tip 351 and a flexible drive shaft 352.

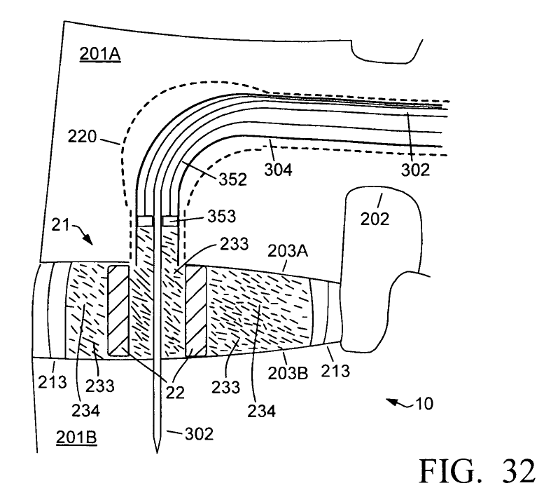

In the FIG. 20-21 embodiment, the plurality of supports 22 includes a plurality of holes 34. The holes 34 facilitate ingrowth of new bone into the plurality of supports 22. As described in connection with FIG. 32, bone graft material 233 that is morselized or flowable may be introduced into the expandable implant 21 and/or may be introduced between the endplates 203A and 203B, using a catheter. The bone graft material 233 may enter the holes 34. In another embodiment, there may be a hole 34 in support first end 23 or support second end 24. In another embodiment, a hole 34 may extend through an individual support 22 from support first end 23 to support first end 24, the hole 34 making a continuous passage from first endplate 203A to second endplate 203B. In another embodiment, the plurality of supports 22 may include a groove 35 (FIG. 24).

FIG. 22 is a perspective view of the central element 70 of the system 10 of FIG. 20. Central element 70 has a central element height 76 that is approximately equal to the support height 26. In another embodiment, central element height 76 may be greater than or less than support height 26.

In the FIG. 20-22 embodiment, central element 70 includes a wall 73 and a lumen 74, and the wall 73 includes a plurality of holes 71. The lumen 74 and the holes 71 facilitate ingrowth of new bone into central element 70, similar to holes 34 in the plurality of supports 22, as described in connection with FIG. 20. Bone graft material 233 that is morselized or flowable may be introduced into lumen 74, as described in connection with FIG. 32.

In another embodiment, central element 70 may be entirely solid or may be mainly solid with a relatively small groove 35 or hole 34, such a a cavity or surface depression. In another embodiment, central element 70 may comprise bone or bone graft substitute. For example, central element 70 may be a plug of bone (a structural autograft or structural allograft).

In the embodiment of FIGS. 20-22, lumen 74 in central element 70 serves as a passage for a guidewire 302. In another embodiment, guidewire 302 may be omitted and central element 70 may be advanced through channel 220 using a steerable driver tool that does not rely upon a guidewire 302. A steerable driver tool may employ a steering mechanism such as a set of telescoping tubes or a tension wire, as described in connection with FIGS. 53, 57, and 58.

FIG. 23 is a section view of a support 22 in an expandable implant 21 (c.f. FIG. 21) in which the means for linking 40 is at least partially insertable within a hole 34 or a groove 35 (FIG. 24) in the plurality of supports 22, in accordance with an embodiment. In the FIG. 23 embodiment, the means for linking 40 is a string 43. In the section view of FIG. 23, the plane of section passes through support first end 23 and support second end 24 and lateral surfaces 33. In the FIG. 23 embodiment, the string 43 is compressed to a rippled or wavelike form within holes 34 when expandable implant 21 is in the unexpanded condition. When the plurality of supports 22 move apart to the expanded condition, tension causes string 43 to be pulled out from holes 34 and become more straight.

FIG. 24 is a section view of two supports 22 in an expandable implant 21 in which the means for linking 40 is at least partially insertable within a hole 34 or a groove 35 in the plurality of supports 22, in accordance with an embodiment. In the section view of FIG. 24, the plane of section is perpendicular to support axis 25. In the FIG. 24 embodiment, the means for linking 40 is a string 43 that is folded for insertion within grooves 35 in adjacent supports 22 in the unexpanded condition. When the plurality of supports 22 move apart to the expanded condition, tension causes string 43 to be unfolded and pulled out of groove 35.

In another embodiment, elongate member 41 (eg a string 43 or a wire 47) may be attached at a peripheral surface 32 of the plurality of supports 22, so that the means for linking 40 (elongate member 41 or string 43) surrounds the plurality of supports 22. In another embodiment, elongate member 41 may be attached at a central surface 31. In another embodiment, elongate member 41 (eg a string 43) may be at least partially insertable within a groove 35 that is located at a peripheral surface 32 or a central surface 31 of the plurality of supports 22. In another embodiment, elongate member 41 (eg a string 43 or a wire 47) may be at least partially insertable within a groove 35 that is located at the support first end 23 or at the support second end 24.

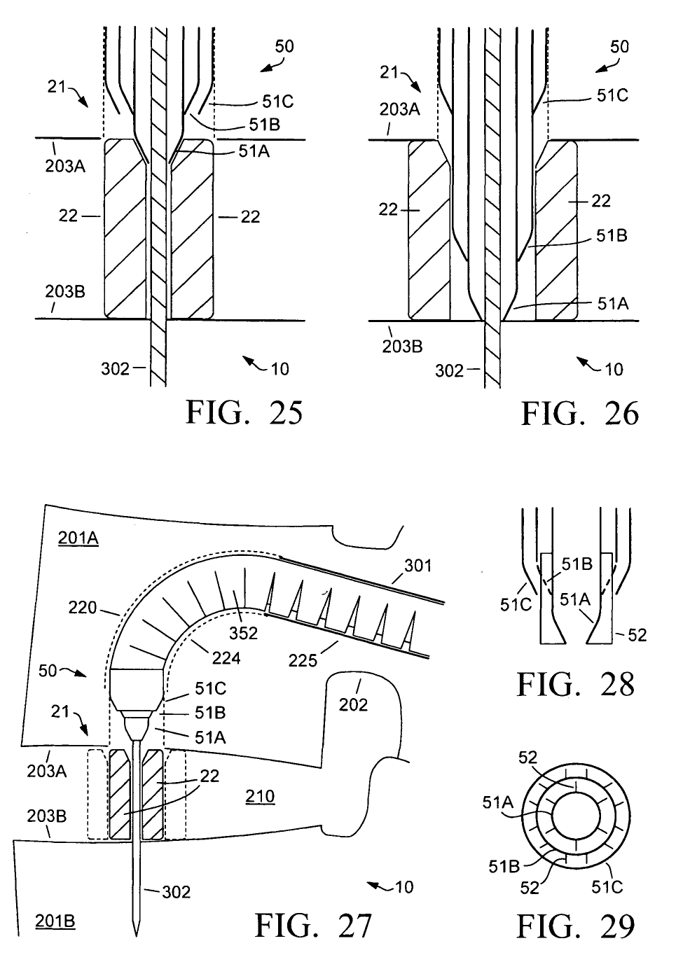

FIG. 25 is a partial section side view of an interbody implant system 10 for use in a spine that includes a first vertebra 201A and a second vertebra 201B, the system 10 comprising an expandable implant 21 and means 50 for expanding the expandable implant 21, in which the means for expanding 50 comprises a plurality of wedges 51A-51C, in accordance with an embodiment. The plurality of wedges 51A-51C is insertable between the plurality of supports 22. In the FIG. 25 embodiment, the plurality of wedges 51 includes a first wedge 51A and a second wedge 51B and a third wedge 51C.

The first wedge 51A is dimensioned to be locatable at least partially within the second wedge 51B, and the second wedge 51B is dimensioned to be locatable at least partially within the third wedge 51C. In the FIG. 25 embodiment, the plurality of wedges 51A-51C is a set of concentric truncated cones. FIG. 25 depicts the system 10 when the first wedge 51A is starting to insert between the plurality of supports 22, when the expandable implant 21 is in the unexpanded condition. FIG. 26 is a partial section side view of the system 10 of FIG. 25 during the inserting of the plurality of wedges 51A-51-C between the plurality of supports 22, when the expandable implant 21 is in a partially expanded condition.

FIG. 27 is a partial section side view of the system 10 of FIG. 25, the view being taken prior to inserting the plurality of wedges 51A-51-C between the plurality of supports 22, in accordance with an embodiment. In the FIG. 25-27 embodiment, the means for expanding 50 includes a plurality of concentric tubular flexible drive shafts 352, each of the plurality of wedges 51A-51C being attached to a separate flexible drive shaft 352. Wedges 51A-51C are inserted sequentially between the plurality of supports 22, with first wedge 51A inserted first, second wedge 51B inserted second, and third wedge 51C inserted third.

In one embodiment, the expandable implant 21 in the FIG. 25-27 embodiment may be advanced through channel 220 by a separate flexible driver 350 which is withdrawn prior to advancing of means for expanding 50. In another embodiment, the separate flexible driver 350 is omitted, and the means for expanding 50 presses against expandable implant 21 to advance it through channel 220. In such an embodiment, the distal (leading) end of first wedge 51A is retracted so that it does not extend beyond second wedge 51B and third wedge 51C, to prevent insertion of first wedge 51A between the plurality of supports 22 during the advancing through channel 220.

In the embodiment of FIG. 27, channel 220 is angled upward in pedicle region 225. The upward angle causes lengthening of central region 224, resulting is a large radius of curvature for central region 224. The large radius of curvature may facilitate alignment of an expandable implant 21 or a central element 70 relative to first endplate 203A. Alignment perpendicular to first endplate 203A may be especially challenging for any element or component that is relatively long. An alternative approach to alignment using a channel 220 with a large central region 224 is described in connection with FIG. 30.

FIG. 28 is a partial section side view of a means for expanding that comprises a plurality of wedges 51A-51C and a plurality of fins 52, in accordance with an embodiment. The plurality of fins 52 on wedge 51 may help to prevent rotation or twisting of the plurality of supports 22 while the supports 22 are forced to move apart by first wedge 51A. Second wedge 51B includes a plurality of slots to accommodate the fins 52 when first wedge 51A is retracted within second wedge 51B. FIG. 29 is a section view of a means for expanding that comprises a plurality of wedges 51A-51C and a plurality of fins 52, in accordance with an embodiment. The plane of section is perpendicular to a central axis for the plurality of wedges 51A-51C. The FIG. 29 embodiment is similar to that of FIG. 28, except that second wedge 51B also includes fins 52.

In another embodiment, means for expanding, equivalent to means 50 in FIG. 25-29, may comprise a plurality of fins 52, the plurality of fins 52 having a plurality of tips 55 that are insertable between the plurality of supports, each fin 52 having a proximal segment, and a wedge 51 that is insertable between the proximal segments. In such an embodiment, a fin 52 may be V-shaped or U-shaped in cross-section, with the V-shape or U-shape contacting a central surface 31 or lateral surfaces 33 of a support 22. Forcing apart of the proximal segments by the wedge 51 causes the fins 52 to move apart, which in turn causes the plurality of supports 22 to move apart to an expanded condition.

FIG. 30 is a partial section side view of an interbody implant system 10 for use in a spine that includes a first vertebra 201A and a second vertebra 201B, the system 10 comprising an expandable implant 21, the expandable implant 21 comprising a plurality of supports 22 and a central element 70 that is insertable between the plurality of supports 22 when the expandable implant 21 is in the expanded condition, in accordance with an embodiment. FIG. 31 is a perspective view of the central element 70 of the system 10 of FIG. 30. In the FIG. 30-31 embodiment, the central element 70 has a central element diameter 75 that is configured to permit passage of the central element 70 through the channel 220.

In FIG. 30, central element 70 is depicted at two different times as it advances through channel 220. At a first time, central element 70 (70A) is positioned within central region 224. At a second time, central element 70 (70B) is positioned inserted between the plurality of supports 22 and extending into second vertebra 201B.

The central element 70 in the FIG. 30 embodiment is similar to the central element 70 in the FIG. 20 embodiment, but with several differences. In the FIG. 30 embodiment, the central element 70 has a central element height 76 that is greater than the support height 26. In the FIG. 30 embodiment, the central element 70 includes means for anchoring 77 in the first vertebra 201A or the second vertebra 201B. In the FIG. 30 embodiment, the means for anchoring 77 is a thread 102, and central element 70 is anchored in second vertebra 201B.

For anchoring central element 70 in second vertebra 201B, a hole may be formed in second vertebra 201B using a drilling tool, prior to advancing expandable implant 21 through channel 220. Central element 70 may be advanced through channel 220 using a flexible driver 350 as in FIG. 6 or FIG. 20.

In another embodiment, a central element 70 that includes a means for anchoring 77 in a vertebra 201 may have a central element height 76 that is less than or equal to the support height 26. In another embodiment, central element 70 may include a first means for anchoring 77 and a second means for anchoring 77 so that central element 70 may be anchored in both the first vertebra 201A and the second vertebra 201B. In another embodiment, means for anchoring 77 may be any type of expansion anchor, and central element 70 may include any type of protrusion 101 such as a ridge 103 for digging into the vertebra 201.

In another embodiment, a central element 70 may comprise a first central element 70A and a second central element 70B, wherein the first central element 70A is positioned adjacent the support first end 23 and the second central element 70B is positioned adjacent the support second end 24. In one such embodiment, the central element 70 may include plural means for anchoring, in which the first central element 70A includes a first means for anchoring and the second central element 70B includes a second means for anchoring, the first central element 70A being anchored in the first vertebra 201A, and the second central element 70B being anchored in the second vertebra 201B.

Central element 70 in the FIG. 30 embodiment is fairly long; in other words, central element height 76 (FIG. 31) is large. The large length or height 76 of central element 70 raises issues with respect to guidewire 302 and with respect to aligning of central element 70. Guidewire 302 is curved within central region 224 of channel 220. To accommodate the curvature of guidewire 302, wall 73 includes a slot 72 that intersects a central element first end 78 or a central element second end 79 for the central element 70. Guidewire 302 may pass through slot 72, as indicated by the overlap of central element 70A and guidewire 302 that is depicted in FIG. 30.

In another embodiment, a driver tip 351 (FIG. 6) for a driver tool 350 may be insertable within lumen 74 of central element 70 (FIG. 31), rather than pressing against first end 78 of central element 70 as in the FIG. 20 embodiment. In such an embodiment, slot 72 may be made wide to accommodate a flexible drive shaft 352 for the driver tool 350.

Central element 70 may need to be aligned properly relative to the first endplate 203A and the plurality of supports 22, and the large length or height 76 may interfere with alignment of central element 70. The FIG. 30 embodiment employs a channel 220 similar to that of FIG. 6, with a central region 224 having a channel diameter 221 that is greater than the channel diameter 221 for the pedicle region 225 or the endplate region 232. The large channel diameter 221 in the central region 224 facilitates aligning the long central element 70 relative to the first endplate 203A and the plurality of supports 22.

As described herein in connection with FIG. 6 and FIG. 30, a channel 220 may have a variable diameter. In one embodiment, the channel diameter 221 for the central region 224 is greater than the channel diameter 221 for the pedicle region 225 and the channel diameter 221 for the central region 224 is greater than the channel diameter 221 for the endplate region 232.

FIG. 32 is a partial section side view of an interbody implant system 10 for use in a spine that includes a first vertebra 201A and a second vertebra 201B, the system 10 comprising an expandable implant 21 and a catheter 304 for introducing bone graft material 233 into the expandable implant 21 when the expandable implant 21 is in the expanded condition, in accordance with an embodiment. Catheter 304 is at least partially insertable within channel 220. In the FIG. 32 embodiment, the bone graft material 233 is morselized or flowable, the bone graft material 233 comprising bone or bone graft substitute. In the FIG. 32 embodiment, a plunger 353 attached to a flexible shaft 352 within catheter 304 serves to press the bone graft material 233 into expandable implant 21.

In the FIG. 32 embodiment, at least a portion of the bone graft material 233 is introduced between the plurality of supports 22. In the FIG. 32 embodiment, the means for linking permits extruding of at least a portion of the bone graft material 233 to a location 234 that is external to the expandable implant 21, the location 234 being between the first endplate 203A and the second endplate 203B. To permit extruding to a location 234 that is external, the means for linking may be any type of means for linking that does not make a tight seal between individual supports 22. For example, means for linking may be an elongate member 41, or a sheet 42 with plural openings 49 (FIG. 21, FIG. 5).

Placement of bone graft material 233 both within expandable implant 21 and at a location 234 that is external to expandable implant 21 may facilitate fusion of vertebra 201. In the example depicted in FIG. 32, the annulus 213 of spinal disc 210 is still at least partially intact and serves to help retain the bone graft material 233 between the first endplate 203A and the second endplate 203B. In another embodiment, the bone graft material 233 may be confined between the plurality of supports 22 with no extrusion to an external location 234.

In another embodiment, an interbody implant system 10 may comprise an expandable implant 21 and a catheter 304 for introducing bone graft material 233 between the first endplate 203A and the second endplate 203B, the catheter 304 being at least partially insertable within the channel 220. In such an embodiment, the bone graft material 233 is morselized or flowable, the bone graft material 233 comprising bone or bone graft substitute. In such an embodiment, the bone graft material 233 may be introduced before the advancing of the expandable implant 21, or before the expanding of expandable implant 21 to an expanded condition, or the bone graft material 233 may be introduced through a first channel 220A with a second channel 220B being used for advancing of expandable implant 21.

In another embodiment, an interbody implant system 10 may comprise an expandable implant 21 and means for expanding the expandable implant 21, wherein the means for expanding comprises means for pressing bone graft material 233 into the expandable implant 21, the means for pressing being at least partially insertable within the channel 220. In such an embodiment, the bone graft material 233 exerts force upon the plurality of supports 22 or the means for linking so that the plurality of supports 22 move apart from one another to an expanded condition. In such an embodiment, the bone graft material 233 is morselized or flowable, the bone graft material 233 comprising bone or bone graft substitute. In such an embodiment, the means for pressing may be a plunger 353 attached to a flexible shaft 352 within catheter 304, similar to that depicted in FIG. 32.

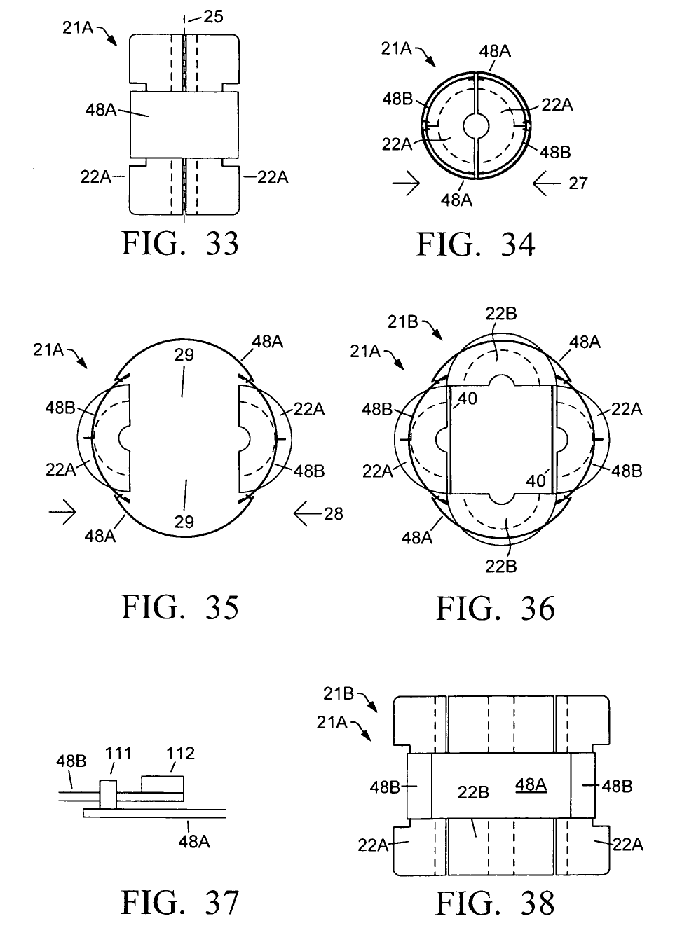

FIG. 33 is a side view of an expandable implant 21A, for an interbody implant system 10 like those described above, in which the means for linking comprises a spring 48, in accordance with an embodiment. FIG. 34 is a section view of the expandable implant 21A of FIG. 33 when the expandable implant 21A is in an unexpanded condition. In the FIG. 33-34 embodiment, the plurality of supports 22A comprises two supports 22A. In the FIG. 33-34 embodiment, spring 48 comprises a plurality of springs 48 that includes a pair of outer springs 48A and a pair of inner springs 48B. Each outer spring 48A and each inner spring 48B is a resilient member having an arc shape. The arc shaped springs 48A and 48B wrap around the middle of the plurality of supports 22A. Springs 48A and 48B are slidably connected using a connector 111 and a stop 112, as detailed in FIG. 37.

FIG. 35 is a section view of the expandable implant 21A of FIG. 33 when the expandable implant 21A is in an expanded condition. The springs 48A-B are under compression when expandable implant 21A is in an unexpanded condition, as depicted in FIG. 34. When the springs 48A-B are released from compression the expandable implant 21A may assume an expanded condition, as depicted in FIG. 35. When the springs 48A-B are released from compression, the radius of curvature increases for each spring 48A-B, and the outer springs 48A slide relative to the inner springs 48B. When the plurality of supports 22A move apart from one another, gaps 29 are created between the plurality of supports 22A.

In another embodiment similar to that of FIGS. 33-35, the system 10 may further comprise a means for expanding such as a wedge 51, as described above. The means for expanding may be used to expand the plurality of supports 22A, in addition to any expanding that results from release of compression. Release from compression may not be sufficient to expand the expandable implant 21A, because friction between the plurality of supports 22A and the endplates 203A-B (e.g. FIG. 32) may hinder movement of the plurality of supports 22A.

The springs 48 may be held under compression using any of the means for holding together the plurality of supports 22 that are described following the description of FIG. 18, such as an easily severable band or a retractable sleeve or a plurality of retractable prongs. Retraction of the sleeve or prongs serves to release the springs 48A-B from compression; in such an embodiment, the means for expanding the expandable implant 21 may comprise the mechanism that retracts the sleeve or prongs.

The pair of supports 22A may be made from bone graft material, including bone or bone graft substitute, or from conventional materials. Similarly, the pair of supports 22B that is depicted in the embodiment of FIGS. 36 and 38 may be made of bone graft material, including bone or bone graft substitute, or from conventional materials.

FIG. 36 is a section view of an expandable implant 21A-21B, for an interbody implant system 10, in which the expandable implant 21A-21B comprises a first expandable implant 21A and a second expandable implant 21B, where the second expandable implant 21B is insertable between the plurality of supports 22A for the first expandable implant 21A when the first expandable implant 21A is in the expanded condition, in accordance with an embodiment. FIG. 38 is a side view of the expandable implant 21A-21B of FIG. 36. In the embodiment of FIGS. 36 and 38, first expandable implant 21A is essentially the same as expandable implant 21A in the FIG. 33-35 embodiment.

Second expandable implant 21B comprises a plurality of supports 22B and a means for linking 40. In the embodiment of FIGS. 36 and 38, at least a majority of the plurality of supports 22B for the second expandable implant 21B are dimensioned to be insertable into a plurality of gaps 29 between the plurality of supports 22A for the first expandable implant 21A in the expanded condition. The second expandable implant 21A is inserted between the plurality of supports 22A and is then expanded with insertion of the plurality of supports 22B into gaps 29. The embodiment of FIG. 19 is another example of an expandable implant 21 that comprises a first expandable implant 21A and a second expandable implant 21B, where the second expandable implant 21B is insertable between the plurality of supports 22A for the first expandable implant 21A when the first expandable implant 21A is in the expanded condition.

In another embodiment, the second expandable implant 21B may be capable of pressing outward on the first expandable implant 21A for causing a further moving apart of the plurality of supports 22A for the first expandable implant 21A, wherein the further moving apart causes an increase in the expanded diameter 28 for the first expandable implant 21A. In such an embodiment, the second expandable implant 21B serves as an additional means for expanding 50. In such an embodiment, the direction of motion for the supports 22B may be parallel to the direction of motion for the supports 22A, rather than a perpendicular direction of motion as in the FIG. 36 embodiment.

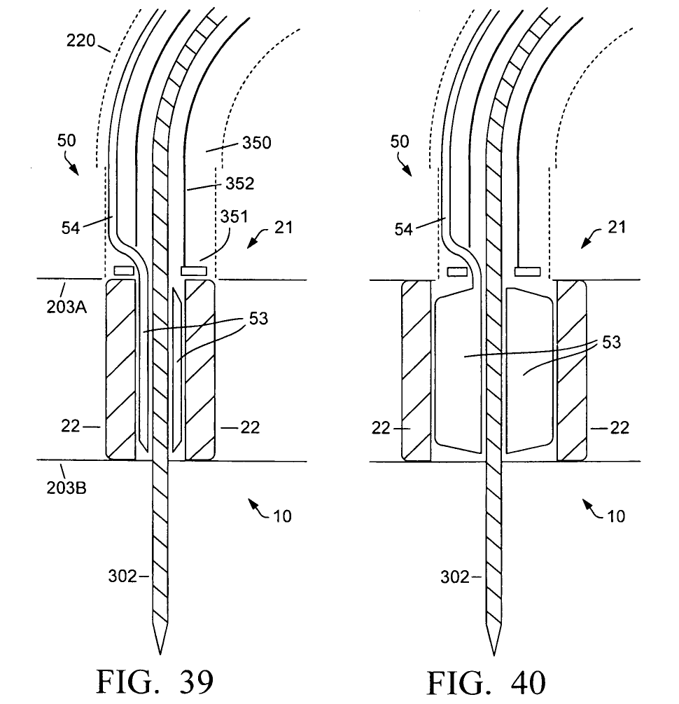

FIG. 39 is a partial section side view of an interbody implant system 10 for use in a spine that includes a first vertebra 201A and a second vertebra 201B, the system comprising an expandable implant 21 and means 50 for expanding the expandable implant 21, in which the means for expanding 50 comprises a balloon 53 and an inflation line 54 that is connected to the balloon, in accordance with an embodiment. FIG. 40 is a partial section side view of the system of FIG. 33 when the balloon 53 is inflated and the expandable 21 implant is in the expanded condition.

Expandable implant 21 and means for expanding 50 may be advanced together through channel 220 using a flexible driver 350 that comprises a driver tip 351 and a flexible drive shaft 352. Alternatively, expandable implant 21 may be advanced first, and then means for expanding 50 may be advanced and inserted between the plurality of supports 22. The plurality of supports 22 is positioned between the first endplate 203A and the second endplate 203B, and then the balloon is inflated to cause the plurality of supports 22 to move apart from one another. Balloon 53 may be a balloon of a type that is used, for example, in angioplasty or for other types of tissue dilation. In the FIG. 39-40 embodiment, balloon 53 includes a passage for a guidewire 302. A balloon 53 that includes a passage for a guidewire 302 is described in U.S. Pat. No. 5,578,009 issued to Kraus.

It may be advantageous to prevent balloon 53 from extending into gaps 29 between the plurality of supports 22, so that balloon 53 exerts force primarily against central surfaces 31 of the plurality of supports 22, as shown for example in FIG. 10. To prevent balloon 53 from extending into gaps 29, means for linking may be positioned at central surface 31, and means for linking may be, for example, a sheet 42 or a stent 45 or a mesh 46. A stent 45 may be selected to be taller before expanding in diameter and thus to become shorter as it expands. If the means for linking in the FIG. 39-40 embodiment is a stent 45 positioned at central surfaces 31, the stent 45 may initially be taller than expandable implant 21 and may extend into the endplate region 232 of channel 220.

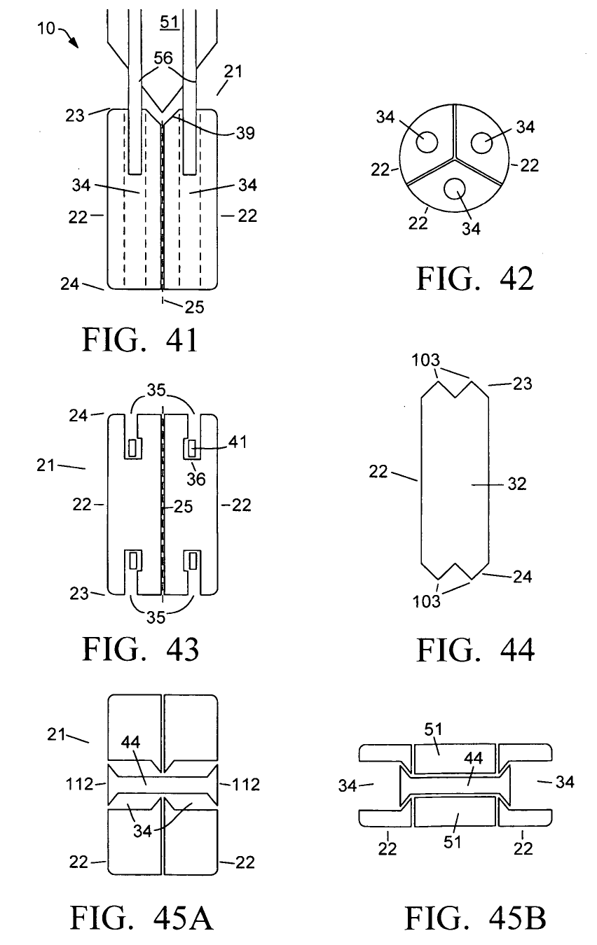

FIG. 41 is a partial section side view of an interbody implant system 10 comprising an expandable implant 21 in which the plurality of supports 22 includes a hole 34 that extends from support first end 23 to support second end 24, in accordance with an embodiment. FIG. 42 is a section view of the expandable implant 21 of the system 10 of FIG. 41, with the plane of section perpendicular to support axis 25. In the FIG. 41-42 embodiment, the plurality of supports 22 comprises three supports 22 and a notch 39 at support first end 23. Each support 22 includes a hole 34 that extends from support first end 23 to support second end 34. Holes 34 may facilitate ingrowth of bone.

In the FIG. 41-42 embodiment, the means for expanding is a wedge 51. A set of prongs 56 extends through wedge 51 and into the plurality of supports 22, with one prong 56 inserted into each hole 34. The expandable implant 21 may be advanced through a channel 220 together with the wedge 51 and the prongs 56 using a steerable drive shaft that uses any of the steering mechanisms described in connection with FIGS. 53-55 and FIGS. 57-58. Prior to expanding of expandable implant 21, prongs 56 may be retracted into wedge 51. Prongs 56 serve as a means for holding together the plurality of supports 22 and also as a means for guiding the position of the expandable implant 21.

FIG. 43 is a section view of an expandable implant 21, for an interbody implant system 10, in which the plurality of supports 22 includes a groove 35 and the means for linking, e.g. 41, is at least partially insertable into the groove 35, in accordance with an embodiment. Each support 22 includes a first groove 35 that intersects support first end 23 and a second groove 35 that intersects supports second end 24. Grooves 35 extend between the lateral surfaces 33 of a support 22. In the FIG. 43 embodiment, each groove 35 includes an enlarged region 36, and the means for linking is an elongate member 41. Elongate members 41 may be inserted into first groove 35 from support first end 23 and may be inserted into second groove 35 from support second end 24.

FIG. 44 is a side view of a support 22 for an expandable implant 21 in which at least one of the plurality of supports 22 includes a ridge 103 at the support first end 23 or the support first end 24, in accordance with an embodiment. The view is towards the peripheral surface 32. In the FIG. 44 embodiment, support 22 includes two ridges 103 at support first end 23 and also includes two ridges 103 at support second end 24. Ridges 103 may dig into endplates 203 at the plurality of supports 22 moves apart and may help to stabilize or anchor the plurality of supports 22.

FIG. 45A is a section view of an expandable implant 21 in which the means for linking comprises an elongate member, like member 41 in FIG. 43, that comprises a rod 44, in accordance with an embodiment. FIG. 45B is a section view of the expandable implant 21 of FIG. 45A, with the plane of section perpendicular to support axis 25 (shown in FIG. 41). In the FIG. 45A-45B embodiment, the plurality of supports 22 comprises two supports 22, and each support 22 has a rectangular cross sectional shape, as depicted in FIG. 45B. Rod 44 is at least partially insertable within a hole 34 in the plurality of supports 22. Rod 44 includes a stop 112 at each end of rod 44. When the plurality of supports 22 moves apart to the expanded condition, the stops 112 retain the ends of rod 44 within the plurality of supports 22. In the FIG. 45A-45B embodiment, the means for expanding is a wedge 51. Wedge 51 is divided into two parts with rod 44 fitting between the two parts of wedge 51 as wedge 51 is inserted between the plurality of supports 22.

In another embodiment, not illustrated, the means for linking may be a spring, similar to the spring in FIGS. 33-38, in which the spring comprises a helical coil. Such an embodiment may include two supports, similar to supports 22 in the FIG. 45A-45B embodiment. The spring (the helical coil) may be at least partially inserted into a hole in each of the two supports. In such an embodiment, the spring (the helical coil) would be positioned perpendicular to the central surfaces 31 of the supports 22, similar to the positioning of the rod 44 in the FIG. 45A-45B embodiment.

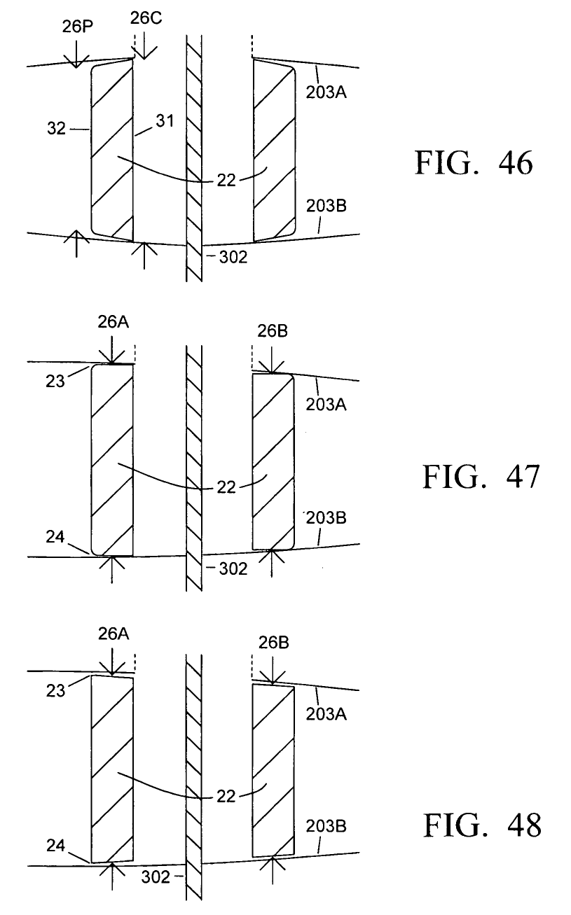

FIG. 46 is a partial section side view of an interbody implant system in which, for at least a majority of the plurality of supports 22, the support height 26C for a central portion of the support 22 is greater than the support height 26P for a peripheral portion of the support 22, in accordance with an embodiment. The FIG. 46 embodiment may facilitate distraction of vertebrae 201 (marked by their opposing surfaces 203A, B). The central portion is the portion near the central surface 31, and the peripheral portion is the portion near the peripheral surface 32. The plurality of supports 22 have a sloping support first end 23 and/or a sloping support second end 24 (as labelled in FIGS. 47 and 48). As the plurality of supports 22 in the device of FIG. 46 move apart, the peripheral portion, which has a smaller support height 26P, slides between the endplates 203A, B. With further moving apart of the plurality of supports 22, the central portion, which has a larger support height 26C, slides between the endplates 203.

FIG. 47 is a partial section side view of an interbody implant system 10 in which the support height 26 differs among the plurality of supports 22, in accordance with an embodiment. The FIG. 47 embodiment may help to achieve or maintain lordosis of vertebrae 201. In the FIG. 47 embodiment, the support height 26A for one of the supports 22 is greater than the support height 26B for another of the supports 22. FIG. 48 is a partial section side view of an interbody implant system in which the support height differs among the plurality of supports, in accordance with an embodiment. The FIG. 48 embodiment is similar to the FIG. 47 embodiment, except that the support first end 23 and support second end 24 are sloping in the FIG. 48 embodiment.

### Table 1

Table 1 describes a method for treating a spine, the method comprising a set of steps (a)-(d) that are listed in Table 1, in accordance with an embodiment. Table 1 is illustrated in FIGS. 49-51, FIGS. 53-55 and FIGS. 57-58

A method for treating a spine, the spine including a first vertebra 201A and a second vertebra 201B, the first vertebra 201A having a first endplate 203A that is adjacent a spinal disc 210, the second vertebra 201B having a second endplate 203B that is adjacent the spinal disc 210, the first vertebra 201A having a body 204 and a pedicle 202, the method comprising:

(a) forming a channel 220 that extends through the first vertebra 201A, wherein the channel 220 extends through the pedicle 202 and through the first endplate 203A, the channel 210 having a channel diameter 221, the channel 220 having a pedicle region 225, a central region 224, and an endplate region 232, wherein the channel diameter 221 for the central region 224 is greater than the channel diameter 221 for the pedicle region 225 and the channel diameter 221 for the central region 224 is greater than the channel diameter 221 for the endplate region 232;

(b) providing an implant, the implant having an implant diameter, wherein the implant diameter is configured to permit passage of the implant through the pedicle region 225 and through the endplate region 232;

(c) introducing the implant into the pedicle region 225; and

(d) advancing the implant through the channel 220, wherein at least a portion of the implant advances at least to the first endplate 203A.

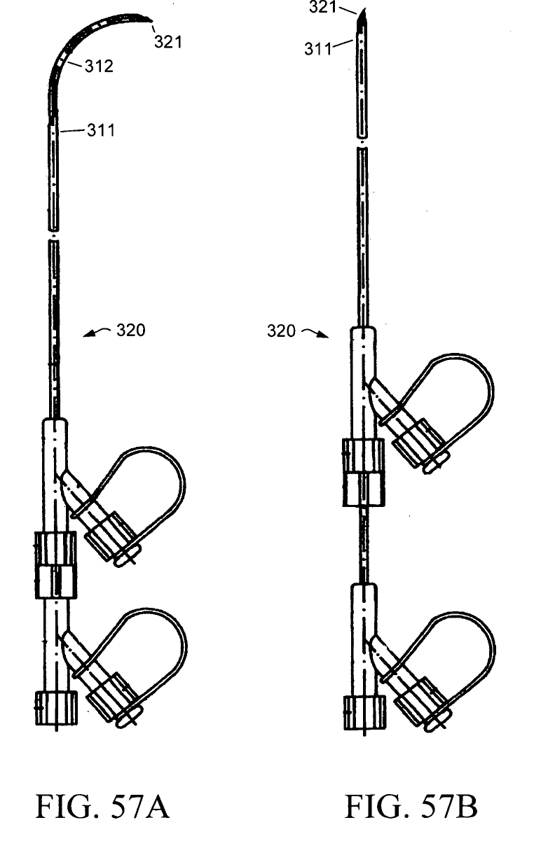

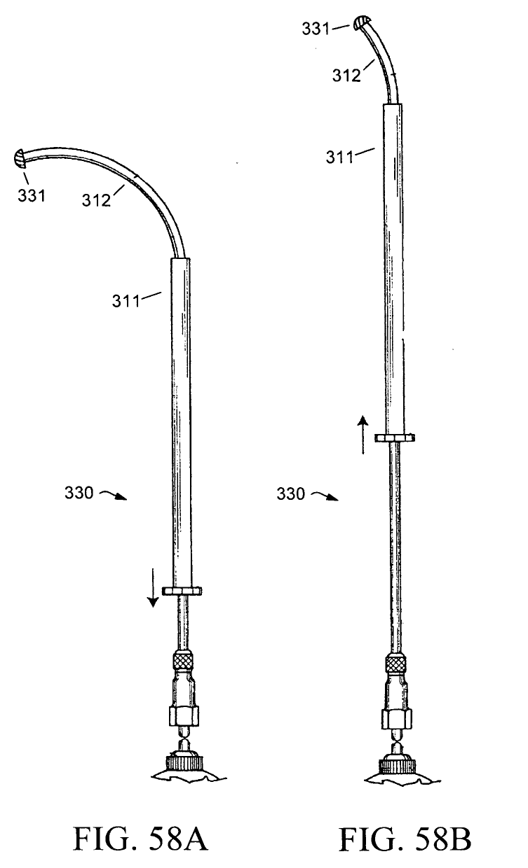

The channel forming step (step a) may be performed as described in connection with FIGS. 49-51 and FIGS. 53-55 and FIGS. 57-58. FIGS. 53-55 depict general aspects of forming a channel 220, and FIGS. 57-58 depict steerable tools that may be used in forming a channel 220. FIGS. 49-51 depict method embodiments for forming a variable diameter channel 220. The methods and tools described in connection with FIGS. 53-55 and FIGS. 57-58 may be used, for example, to form predecessor channels in the FIG. 49-51 embodiments.

With respect to the providing step (step b), a variable diameter channel 220 may be used with many types of implant. As indicated in step (b), the provided implant has an implant diameter that is configured to permit passage of the implant through the pedicle region 225 and through the endplate region 232. The implant may be an expandable implant 21 such as an expandable implant 21 described herein or the implant may be a non-expandable implant. In one embodiment, for example, a variable diameter channel may be used with a non-expandable implant that includes means 77 for anchoring in first vertebra 201A or second vertebra 201B; such an implant may be similar to, for example, the central element 70 in the FIG. 30-31 embodiment which includes a thread 102 for anchoring. In another embodiment, a variable diameter channel 220 may be used with a non-expandable implant that comprises more than one component, with one component anchored in a first vertebra 201A and another component anchored in a second vertebra 201B.

With respect to step (c), the implant may be introduced into the pedicle region 225 using a posterior approach 240 as depicted in FIGS. 1-2. In one embodiment, the surgical approach is percutaneous and employs a cannula 301 such as that depicted in FIG. 27.

With respect to step (d), the implant may be advanced through the channel 220 using a flexible driver 350 and a guidewire 302, or using a steerable driver tool, or the implant may be advanced together with another element such as a means for expanding 50, as described herein in connection with various Figures.

In another embodiment, the method further comprises installing the implant, wherein the installing comprises positioning the implant at least partially within the spinal disc 210 or at least partially within the first vertebra 201A or at least partially within the second vertebra 201B. For example, the implant may be positioned within the spinal disc 210, as in FIGS. 11-12. In another example, the implant may be positioned partially within the spinal disc 210 and partially within the second vertebra 201B, as depicted in FIG. 30. In another example, the implant may be positioned partially or entirely within first vertebra 201A by anchoring the implant using, for example, a thread 102 that serves to retain the implant within, for example, pedicle region 232.

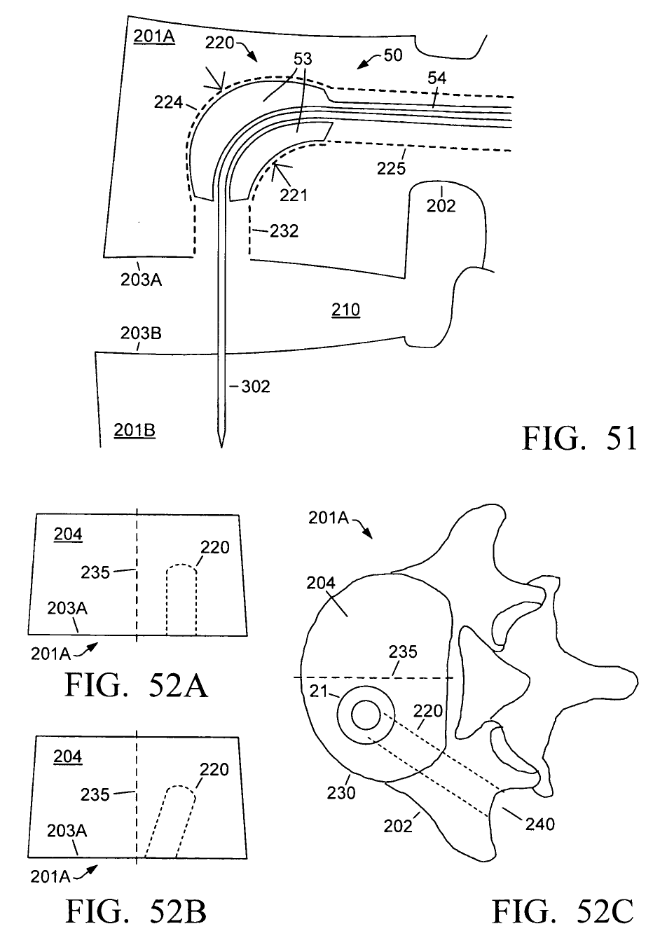

In another embodiment, the forming comprises creating a predecessor channel that extends through the pedicle 202 and through the first endplate 203A, wherein the predecessor channel is coaxial with the channel 220 in at least a portion of the pedicle region 225 and the predecessor channel is coaxial with the channel 220 in at least a portion of the endplate region 232; and enlarging the central region 224 for the predecessor channel, wherein the enlarging causes the channel diameter 221 for the central region 224 to be greater than the channel diameter 221 for the pedicle region 225 and the enlarging causes the channel diameter 221 for the central region 224 to be greater than the channel diameter 221 for the endplate region 232. The embodiment described in the previous sentence includes embodiments such as those depicted in FIGS. 49 and 51, which are described in subsequent paragraphs.

In another embodiment that is depicted in FIGS. 49A and 49B, the enlarging comprises cutting or abrading the body 204 (labelled in FIG. 52) where it surrounds the central region 224 (FIG. 49, 50,51) of the predecessor channel 220P (FIG. 51). Cutting or abrading, as shown in FIG. 49A, is done using a drill, the drill comprising a steerable drill or a flexible drill 340, the drill comprising a retractable cutting head 343 and a sheath 344, the retractable cutting head 343 being capable of retracting within the sheath 344, the sheath 344 dimensioned to be insertable within the predecessor channel 220P, the retractable cutting head 343 capable of emerging from a distal end of the sheath 344. In a preferred embodiment, FIG. 49B, a cutting head radius 345 for the emerged retractable cutting head 343 is greater than half of the channel diameter 221 for the pedicle region 225.

In another embodiment that is depicted in FIG. 50, the forming comprises creating a first predecessor channel 220F and a second predecessor channel 220S, wherein the second predecessor channel 220S diverges from the first predecessor channel 220F in at least a portion of the central region 224. In the embodiment of FIG. 50, the central region 224 has an oval cross-section.

In another embodiment, the enlarging comprises advancing a dilator in the predecessor channel to a position within the central region 224, and dilating the dilator for displacing cancellous bone of the body 204 that surrounds the central region 224 of the predecessor channel. In one embodiment that is depicted in FIG. 51, the dilator comprises a balloon 53 and an inflation line 54 that is connected to the balloon 53, and the dilating comprises inflating the balloon 53. In another embodiment, the dilator may comprise a wedge. For example, the dilator may comprise a flexible sleeve and a wedge that is insertable within a narrow lumen of the flexible sleeve, the inserting of the wedge forcing the sleeve outward to displace cancellous bone.

The dimensions for the channel 220 and the expandable implant 21 may be selected at least partially based on the size and shape of the vertebrae 201 for the patient to be treated. The dimensions of a vertebra 201, such as pedicle height 205, pedicle width 206, and vertebral body height 219, vary widely between individual humans. Table 2 indicates mean values in millimeters, and ranges for these values, for several dimensions of human lumbar vertebrae L3, L4, and L5. It is understood that the values in Table 2 represent measured values for specific groups of human subjects, and that the actual range of values for dimensions of a vertebra 201 may differ from the range of values indicated in Table 2. The first sacral (S1) vertebra has a vertebral body height 219 that is similar to that of the lumbar vertebrae.

TABLE 2

| | body | pedicle | pedicle | disc |
|---|---|---|---|---|
| | height | width | height | height |
| L3 | 30 | 10 | 15 | 12 |
| | 23-36 | 5-16 | 8-18 | 7-16 |
| L4 | 29 | 13 | 15 | 11 |
| | 22-35 | 9-17 | 9-19 | 5-16 |
| L5 | 28 | 18 | 14 | 11 |
| | 22-35 | 9-29 | 10-19 | 6-16 |

The values for vertebral body height 219 (“body height”) and for disc height are adapted from a journal article by Zhou, S. H., McCarthy, I. D., McGregor, A. H., Coombs, R. R. H., and Hughes, S. P. F., “Geometrical dimensions of the lower lumbar vertebrae—analysis of data from digitised CT images”, Eur. Spine J. 9:242-248, 2000. For the body height for each vertebra L3, L4, and L5, the first line indicates the average of the published mean values for the anterior body height and the posterior body height, and the second line indicates the average of the published range of values for the anterior body height and the posterior body height, each average being rounded to the nearest whole number. The values for pedicle width 206 and pedicle height 205 are adapted from a book entitled “Clinical Biomechanics of the Spine” by White, A. and Panjabi, M., Table 1-6, page 32, J.B. Lippincott Company, 1990. For the pedicle dimensions for each vertebra L3, L4, and L5, the first line indicates the mean value and the second line indicates the range of values. The disc height refers to the height of the spinal disc 210 that is caudal to each vertebra L3, L4, or L5, the disc height being measured at the anterior-posterior midline. For the disc height, the first line indicates the mean value and the second line indicates the range of values, each value being rounded to the nearest whole number.

A normal (undiseased) spine exhibits lordosis in the lumbar region. Thus, the first endplate 203A and the second endplate 203B are slightly angled relative to one another, with a greater spacing between the endplates 203 at the anterior region of spinal disc 210 compared to the spacing at the posterior region of spinal disc 210. Expandable implant 21 may be installed at a location that is somewhat anterior to the anterior-posterior midplane of body 204. Installation at an anterior location may assist maintenance or recreation of lordosis.

As depicted in FIG. 2, a plurality of channels 220 may be formed in a vertebra 201, so that a plurality of expandable implants 21 may be installed. For example, as depicted in FIG. 2, there may be a pair of channels 220, with a channel 220 extending through each pedicle 202. The plurality of expandable implants 21 may be installed symmetrically with respect to a sagittal plane for the vertebrae 201, as depicted in FIG. 2.

In other embodiments, for example as shown in FIG. 52C, a single expandable implant 21 may be installed in a vertebra 201. In one embodiment, a single expandable implant 21 may be installed at an asymmetric position with respect to a sagittal plane for the vertebra 201. Alternatively, a single expandable implant 21 may be installed at a position that is located on or near the sagittal plane for a vertebra 201. FIG. 52A is a section view, from the anterior, of a vertebral body 204 in which a single channel 220 is formed, the channel 220 being located at an asymmetric position with respect to the sagittal plane 235. FIG. 52B is a section view, from the anterior, of a vertebral body 204 in which a single channel 220 is formed, the channel 220 being angled so that it intersects first endplate 203A close to the sagittal plane 235. FIG. 52C is an axial view of a vertebra 201 (lumbar vertebra L5) in which a single channel 220 is formed, with a single expandable implant 21 installed in the vertebra 201 at an asymmetric position with respect to the sagittal plane 235.

For a pair of vertebrae 201 that includes a cepahalad vertebra 201 and a caudal vertebra 201, the channel 220 may be located in the cephalad vertebra 201 as in FIG. 6 or in the caudal vertebra 201 as in FIG. 56. In other words, first vertebra 201A may be the cephalad vertebra 201 as in FIG. 6, or first vertebra 201A may be the caudal vertebra 201 as in FIG. 56. FIG. 56 is a partial section side view of two vertebrae 201A and 201B and a channel 220 formed in the caudal vertebra 201A. System 10 may be used with any vertebra 201 from any region of the spine 200, as long as the dimensions of the vertebra 201 are suitable. For example, the first sacral (S1) vertebra may be the first vertebra 201A or the second vertebra 201B.

Table 3 indicates a method for treating a spine, the method comprising a set of steps (a)-(d) that are listed in Table 3, in accordance with an embodiment.

### Table 3

A method for treating a spine, the spine including a first vertebra 201A and a second vertebra 201B, the first vertebra 201A having a first endplate 203A that is adjacent a spinal disc 210, the second vertebra 201B having a second endplate 203B that is adjacent the spinal disc 210, the first vertebra 201A having a pedicle 202 and a body wall 230, the method comprising:

(a) forming a channel 220 that extends through the first vertebra 201A, wherein the channel 220 extends through the pedicle 202 or the body wall 230 and the channel 220 extends through the first endplate 203A, the channel 220 having a channel axis 222 and a channel diameter 221, the channel axis 222 at the first endplate 203A being oblique or perpendicular to the first endplate 203A;

(b) providing an expandable implant 21, the expandable implant 21 comprising a plurality of supports 22 and means 40 for linking the plurality of supports 22, the plurality of supports 22 being capable of moving apart from one another, the plurality of supports 22 having a support first end 23 and a support second end 24 and a support axis 25 that extends from the support first end 23 to the support second end 24, the plurality of supports 22 having a support height 26, the expandable implant 21 having an unexpanded diameter 27 that is perpendicular to the support axis 25;

(c) advancing the expandable implant 21 through the channel 220, wherein the unexpanded diameter 27 is configured to permit passage of the expandable implant 21 through the channel 220; and

(d) expanding the expandable implant 21 to an expanded condition, wherein the expanding comprises moving the plurality of supports 22 apart from one another, the expandable implant 21 having an expanded diameter 28 when the expandable implant 21 is in the expanded condition, the expanded diameter 28 being perpendicular to the support axis 25,

wherein the expanded diameter 28 is configured to be greater than the channel diameter 221 at the first endplate 203A; and

wherein the support height 26 is configured to permit the support second end 24 to be positioned adjacent the second endplate 203B while the support first end 23 is positioned adjacent the first endplate 203A while the support axis 25 is oriented substantially perpendicular to the first endplate 203A.

The channel forming step (step a) may be performed as described in connection with FIGS. 53-55 and FIGS. 57-58. FIGS. 53-55 depict general aspects of forming a channel 220, and FIGS. 57-58 depict steerable tools that may be used in forming a channel 220. In one embodiment, the channel 220 may extend through the body wall 230 and through the first endplate 203A. In another embodiment, the channel 220 may extend through the pedicle 202 and through the first endplate 203A.

With respect to the providing step (step b), the expandable implant 21 may be any expandable implant 21 similar to those described herein or having the characteristics that are described in detail in connection with FIGS. 3-6.

With respect to step (c), the implant may be advanced through the channel 220 using a flexible driver 350 and a guidewire 302, or using a steerable driver tool, or the implant may be advanced together with another element such as a means for expanding 50, as described herein in connection with various Figures.

With respect to step (d), the expandable implant 21 may be expanded using any suitable means for expanding 50 such as any of the means for expanding 50 that are described herein.

In another embodiment, the forming causes the channel diameter 221 for the central region 224 to be greater than the channel diameter 221 for a pedicle region 225 of the channel 220 and greater than the channel diameter 221 for the endplate region 232 of the channel 220. FIGS. 49-51 depict method embodiments for forming a variable diameter channel 220 such as the channel 220 described in the previous sentence. The methods and tools described in connection with FIGS. 53-55 and FIGS. 57-58 may be used, for example, to form predecessor channels in the FIG. 49-51 embodiments.

In another embodiment, the method further comprises preparing the spinal disc 210 and the first endplate 203A and the second endplate 203B prior to advancing the expandable implant 21 through the channel 220, wherein the preparing comprises removing at least a portion of a nucleus for the spinal disc 210 and abrading the first endplate 203A and abrading the second endplate 203B. The abrading may include removing at least a portion of the external cartilage layer of the first endplate 203A or the second endplate 203B. The preparing may employ a directed jet of water as in cutting devices supplied by HydroCision Corporation of Massachusetts, US. The preparing may employ a cutting device or an enucleation device such as those depicted in FIGS. 31-36 of U.S. Pat. No. 7,318,826 issued to Teitelbaum or those described in U.S. Patent Application Publication No. 2007/0260270 of Assell.

In another embodiment, the expanding further comprises inserting a wedge 51 between the plurality of supports 22. Expanding using a wedge 51 is described in connection with FIGS. 11-13 and FIGS. 25-29. In another embodiment, the expanding further comprises inflating a balloon 53 that is positioned between the plurality of supports 22, as described in connection with FIGS. 39-40. In another embodiment, the expanding further comprises introducing bone graft material 233 through a catheter 304 into the expandable implant 21, the bone graft material 233 being morselized or flowable, the bone graft material comprising bone or bone graft substitute, as described in connection with FIG. 32.

In another embodiment, the method further comprises introducing bone graft material 233 between the first endplate 203A and the second endplate 203B using a catheter 304, the bone graft material 233 being morselized or flowable, the bone graft material comprising bone or bone graft substitute, as described in connection with FIG. 32. In another embodiment, the method further comprises introducing bone graft material 233 through a catheter 304 into the expandable implant 21 when the expandable implant 21 is in the expanded condition, the bone graft material 233 being morselized or flowable, the bone graft material comprising bone or bone graft substitute, as described in connection with FIG. 32.

In another embodiment, the method further comprises inserting a central element 70 between the plurality of supports 22 when the expandable implant 21 is in the expanded condition, the central element 70 having a central element diameter 75 that is configured to permit passage of the central element 70 through the channel 220, as described in connection with FIGS. 20-22 and 30-31. In another embodiment, the method further comprises anchoring the central element 70 in the first vertebra 201A or the second vertebra 201B, as described in connection with FIGS. 30-31.

In another embodiment, the providing further comprises providing a second expandable implant 21B; and the method further comprises advancing the second expandable implant 21B through the channel 220, inserting the second expandable implant 21B between the plurality of supports 22 for the expandable implant 21 when the expandable implant 21 is in the expanded condition, and expanding the second expandable implant 21B, as described in connection with FIG. 19 and FIGS. 36 and 38.

FIGS. 53-55 depict details of the forming of a channel 220, in accordance with an embodiment. In the embodiment depicted in FIGS. 53-55, the first vertebra 201A is the cephalad vertebra 201 of the pair of vertebra 201, and the channel 220 extends in a caudal direction. In another embodiment, the first vertebra 201A may be the caudal vertebra 201 of the pair, in which case the channel 220 would extend in a cephalad direction. FIG. 56 depicts a pair of vertebrae 201 and a channel 220 in which the first vertebra 201A is the caudal vertebra 201 of the pair.

The method embodiment depicted in FIGS. 53-55 is performed using a percutaneous transpedicular posterior approach 240. Other embodiments may use an anterior approach 243 through body wall 230 or a lateral approach 242 through body wall 230. In the depicted embodiment, the channel 220 is curved.

In the transpedicular posterior approach 240 used in the embodiment of FIGS. 53-55, a standard bone drill may be used to drill through the pedicle 202 to the body 204 (see FIG. 52, e.g., for a cross-sectional view and numbering). This initial channel segment corresponds to the pedicle region 225 of what will eventually become channel 220. The channel diameter 221 for the initial channel segment may be selected in relation to the dimensions for the first vertebra 201A, as described in connection with Table 2. A cannula 301 may be inserted into the initial channel segment.

In the embodiment of FIGS. 53-55, a narrow curved pilot channel is formed using a steerable channel forming tool, which in this embodiment is a steerable drilling device 330. The narrow curved pilot channel is a precursor to the curved region 224 of the channel 220. For embodiments that use an anterior approach 243 or a lateral approach 242, the channel 220 begins at a hole drilled in the body wall 230. In the depicted embodiment, the narrow curved pilot channel extends in an anterior and caudal direction, so that upon completion of the forming of the narrow curved pilot channel, the axis at the tip of the steerable channel forming tool is oblique or perpendicular to the first endplate 203A, as depicted in FIG. 53.

The narrow curved pilot channel may stop short of the first endplate 203A or may penetrate the first endplate 203A. The steerable channel forming tool is steered so that the resulting narrow curved pilot channel is oblique or perpendicular to the first endplate 203A.

Various steerable channel forming tools may be used to form the narrow curved pilot channel. FIGS. 57 and 58 depict two examples of steerable channel forming tools. The tools of FIGS. 57 and 58 each include an outer tube 311 that is relatively rigid and an elastic pre-curved tube 312 disposed within the outer tube 311. The elastic pre-curved tube 312 may be advanced and retracted relative to the outer tube 311 in a telescoping manner. Retraction of the elastic pre-curved tube 312 within outer tube 311 causes straightening of the elastic pre-curved tube 312. Advancing of the elastic pre-curved tube 312 so that it extends beyond the outer tube 311 allows the elastic pre-curved tube 312 to regain its curvature, causing the tip of the elastic pre-curved tube 312 to point in a direction that is not aligned with the axis of the outer tube 311, thereby enabling the forming of a narrow curved pilot channel.

The steerable channel forming tool depicted in FIGS. 57A-57B is a steerable needle 320 having a beveled tip 321 at the end of the elastic pre-curved tube 312. FIGS. 57A and 57B are adapted from FIGS. 6 and 7 of U.S. Pat. No. 6,572,593 issued to Daum. The steerable channel forming tool depicted in FIGS. 58A-58B is a steerable drilling device 330 having a drill bit 331 at the end of the elastic precurved tube 312. FIGS. 58A and 58B are adapted from FIGS. 7 and 8 of U.S. Pat. No. 6,740,090 issued to Cragg. A steerable drilling device 330 very similar to that of U.S. Pat. No. 6,740,090 is described in detail in U.S. Pat. No. 7,241,297 issued to Shaolian. Another type of steerable channel forming tool is a tension wire drill such as that depicted in FIGS. 19 and 20 of U.S. Pat. No. 6,740,090. Other types of steerable drilling devices or shavers are described in U.S. Pat. No. 5,851,212 issued to Zirps and in U.S. Pat. No. 6,645,218 issued to Cassidy.

In the embodiment of FIGS. 53-54, the steerable channel forming tool is a steerable drilling device 330 having a drill bit 331 and a flexible drive shaft 332. Flexible drive shaft 332 is a hollow tubular drive shaft capable of receiving a guide wire 302. Drill bit 331 similarly has a passage for a guide wire 302. After the forming of the narrow curved pilot channel, a guide wire 302 is introduced into the lumen of flexible drive shaft 332 and the guide wire 302 is advanced so that it extends through and beyond drill bit 331.

The guide wire 302 has a sharp tip 303. As depicted in FIG. 54, the guide wire 302 is advanced so that the tip 303 penetrates the first endplate 203A, the spinal disc 210, and the second endplate 203B, and then continues further into the body 204 of second vertebra 201B. The steerable drilling device 330 is withdrawn without disturbing the guide wire 302, which remains in place with the guidewire tip 303 poking into second vertebra 203B.

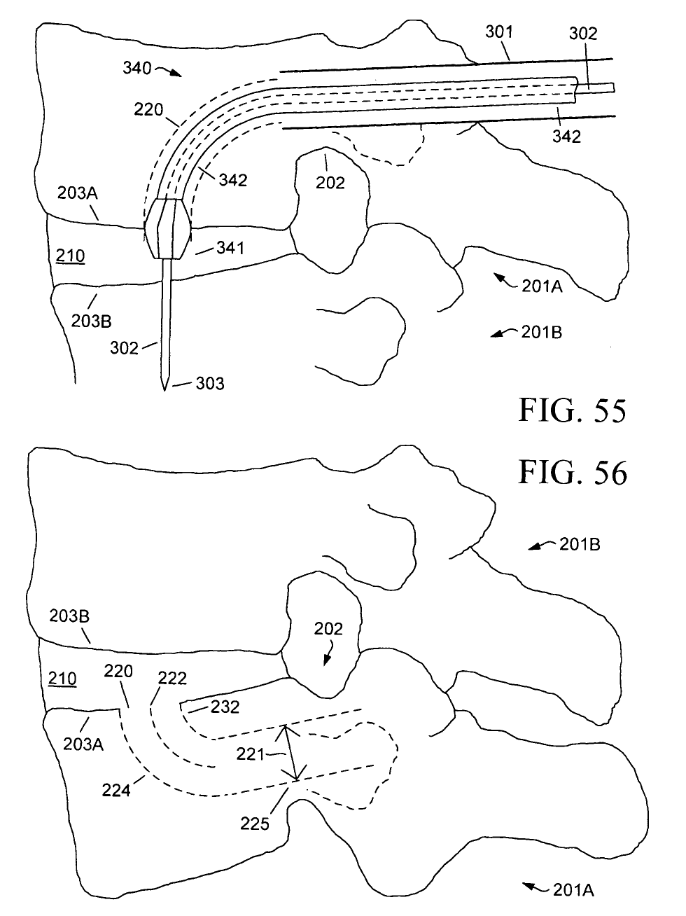

A flexible drill 340 is then introduced into cannula 301 over guide wire 302, as depicted in FIG. 55. The flexible drill 340 has a hollow flexible drive shaft 342 and a cutting head 341 that has a passage for the guide wire 302. The flexible drill 340 may be used to enlarge the narrow curved pilot channel within first vertebra 201A and to extend the channel 220 through the first endplate 203A, as depicted in FIG. 55. In another embodiment, the flexible drill 340 may be used to additionally drill a hole into the second endplate 203B, to enable anchoring of a threaded central element 70 into the second endplate 203B, as depicted in FIG. 30. The flexible drill 340 is withdrawn without disturbing the guide wire 302, which remains in place with the guidewire tip 303 poking into second vertebra 203B.

Forming of a variable diameter channel 220 is described in connection with FIGS. 49-51.

Embodiments described herein may be made from various materials known to be suitable for use in medical devices, including any material that has been approved by the Food and Drug Administration for use in spinal applications. For the plurality of supports 22 and the central element 70, such materials include bone graft material, including bone or bone graft substitute. Such materials include metals such as titanium or stainless steel or cobalt. Such materials include metal alloys such as titanium alloys, including alloys of titanium and stainless steel, and “shape memory” alloys such as nitinol. Such materials include polymers such as polyetheretherketone (“PEEK”). Polymers may be used with or without carbon fiber (to enhance structural strength). Such materials may also include ceramics. The material may be radio opaque or radiolucent. The material for the plurality of supports 22 may be made from a material that is capable of withstanding without significant deformation the force exerted by the means for expanding 50.

The means for linking 40 may be made from various materials, the choice of material depending in part upon the degree of flexibility that is appropriate for a particular means for linking 40. Suitable materials include material used to make a monofilament or braided suture, and include various polymers such as polyester or polyethylene. For a spring 48 or a wire 47, a metal or metal alloy may be used. For a rod 44, a relatively rigid polymer such as PEEK may be used. Several materials may be combined to make a means for linking; for example, braided suture may be embedded in a spaced apart configuration within a sheet 42 that is made from a polymer.

The means for expanding 50 may be made from various materials including those listed above for the plurality of supports 22 and the central element 70. Where the means for expanding 50 comprises a balloon 53, the balloon 53 may be made of materials such as those used in balloons 53 used for dilating tissue or for angioplasty.

As used herein and in the appended claims, the term “thread” 102 means a helical or spiral ridge on a screw, nut, or bolt, or on a cylindrical component such as the central element 70 in the embodiment of FIG. 30-31. As used herein and in the appended claims, the term “ridge” 103 means an elongate protrusion on the surface of a component; the surface having the ridge 103 may be flat or curved.

Although we have described in detail various embodiments, other embodiments and modifications will be apparent to those of skill in the art in light of this text and accompanying drawings. The following claims are intended to include all such embodiments, modifications and equivalents.

What is claimed is:

1. An interbody implant system for use in a spine, the spine including a first vertebra and a second vertebra, the first vertebra having a first endplate that is adjacent a spinal disc, the second vertebra having a second endplate that is adjacent the spinal disc, the system comprising:
an expandable implant, the expandable implant comprising:
a plurality of supports, the plurality of supports being capable of moving apart from one another whereby the expandable implant is in an expanded condition, the plurality of supports having a support first end and a support second end and a support axis that extends from the support first end to the support second end, the plurality of supports having a support height, the expandable implant having an unexpanded diameter that is perpendicular to the support axis, the expandable implant having an expanded diameter when the expandable implant is in the expanded condition, the expanded diameter being perpendicular to the support axis; and
means for linking the plurality of supports, wherein each of the plurality of supports is linked to at least another one of the plurality of supports by the means for linking;
wherein the unexpanded diameter is configured to permit passage of the expandable implant through a channel in the first vertebra, the channel extending at least through the first endplate, the channel having a channel axis and a channel diameter, the channel axis at the first endplate being oblique or perpendicular to the first endplate;
wherein the expanded diameter is configured to be greater than the channel diameter at the first endplate; and
wherein the support height is configured to permit the support second end to be positioned adjacent the second endplate while the support first end is positioned adjacent the first endplate while the support axis is oriented substantially perpendicular to the first endplate.

2. The system of claim 1,
wherein the channel extends through a pedicle for the first vertebra, and wherein the unexpanded diameter is further configured to permit passage of the expandable implant through a pedicle region for the channel.

3. The system of claim 1,
wherein the plurality of supports has a first end surface area for the support first end and a second end surface area for the support second end; wherein the expandable implant in the unexpanded condition has a first end envelope area and a second end envelope area; and wherein the first end surface area is greater than or equal to 50 percent of the first end envelope area and wherein the second end surface area is greater than or equal to 50 percent of the second end envelope area.

4. The system of claim 3,
wherein the first end surface area is greater than or equal to 70 percent of the first end envelope area and wherein the second end surface area is greater than or equal to 70 percent of the second end envelope area.

5. The system of claim 3,
wherein the first end surface area is greater than or equal to 90 percent of the first end envelope area and wherein the second end surface area is greater than or equal to 90 percent of the second end envelope area.

6. The system of claim 1,
wherein a ratio of the expanded diameter to the unexpanded diameter is greater than or equal to 1.75.

7. The system of claim 1, further comprising:
means for expanding the expandable implant.

8. The system of claim 7,
wherein the means for expanding comprises a wedge that is insertable between the plurality of supports.

9. The system of claim 8,
wherein the wedge comprises a fin.

10. The system of claim 8,
wherein the wedge comprises bone or bone graft substitute.

11. The system of claim 8,
wherein the wedge comprises a plurality of wedges, the plurality of wedges including a first wedge and a second wedge, the first wedge dimensioned to be locatable at least partially within the second wedge.

12. The system of claim 7,
wherein the means for expanding comprises:
a plurality of fins, the plurality of fins having a plurality of tips that are insertable between the plurality of supports, each fin having a proximal segment; and
a wedge that is insertable between the proximal segments.

13. The system of claim 7,
wherein the means for expanding comprises a balloon and an inflation line that is connected to the balloon.

14. The system of claim 7,
wherein the means for linking comprises a spring under compression; and wherein the means for expanding comprises means for releasing the spring from compression.

15. The system of claim 7,
wherein the means for expanding comprises:
means for pressing bone graft material into the expandable implant, the means for pressing being at least partially insertable within the channel,
wherein the bone graft material is morselized or flowable, the bone graft material comprising bone or bone graft substitute.

16. The system of claim 1, further comprising:
a catheter for introducing bone graft material between the first endplate and the second endplate, the catheter being at least partially insertable within the channel,
wherein the bone graft material is morselized or flowable, the bone graft material comprising bone or bone graft substitute.

17. The system of claim 1, further comprising:
a catheter for introducing bone graft material into the expandable implant when the expandable implant is in the expanded condition, the catheter being at least partially insertable within the channel,
wherein the bone graft material is morselized or flowable, the bone graft material comprising bone or bone graft substitute.

18. The system of claim 17,
wherein the means for linking permits extruding of at least a portion of the bone graft material to a location that is external to the expandable implant, the location being between the first endplate and the second endplate.

19. The system of claim 1, wherein the expandable implant further comprises:
a central element that is insertable between the plurality of supports when the expandable implant is in the expanded condition, the central element having a central element diameter that is configured to permit passage of the central element through the channel.

20. The system of claim 19,
wherein the central element comprises bone or bone graft substitute.

21. The system of claim 19,
wherein the central element includes a wall and a lumen.

22. The system of claim 21,
wherein the wall includes a hole.

23. The system of claim 21, wherein the wall includes a slot that intersects a central element first end or a central element second end for the central element.

24. The system of claim 19, wherein the central element has a central element height that is less than or equal to the support height.

25. The system of claim 19, wherein the central element has a central element height that is greater than the support height.

26. The system of claim 19, wherein the central element includes means for anchoring in the first vertebra or the second vertebra.

27. The system of claim 19, wherein the central element comprises a first central element and a second central element, wherein the first central element is positioned adjacent the support first end and the second central element is positioned adjacent the support second end.

28. The system of claim 1,
wherein the expandable implant comprises a first expandable implant and a second expandable implant, and wherein the second expandable implant is insertable between the plurality of supports for the first expandable implant when the first expandable implant is in the expanded condition.

29. The system of claim 28, wherein at least a majority of the plurality of supports for the second expandable implant are dimensioned to be insertable into a plurality of gaps between the plurality of supports for the first expandable implant in the expanded condition.

30. The system of claim 28,
wherein the second expandable implant is capable of pressing outward on the first expandable implant for causing a further moving apart of the plurality of supports for the first expandable implant, wherein the further moving apart causes an increase in the expanded diameter for the first expandable implant.

31. The system of claim 1, wherein the plurality of supports comprises at least three supports or at least four supports or at least five supports or at least six supports.

32. The system of claim 1, wherein the plurality of supports includes a hole.

33. The system of claim 1, wherein the plurality of supports includes a groove.

34. The system of claim 1,
wherein the expandable implant includes a passage for a guidewire, the passage extending from the support first end to the support second end; and
wherein at least one of the plurality of supports has a central surface that is at least partially curved relative to the support axis, the at least partially curved central surface defining at least a portion of the passage for the guidewire.

35. The system of claim 1,
wherein the expandable implant includes a passage for a guidewire, the passage extending from the support first end to the support second end; and
wherein the passage is offset from the support axis.

36. The system of claim 1, further comprising:
a guidewire,
wherein a first flexibility for a central portion of the guidewire is greater than a second flexibility for a distal portion of the guidewire, the distal portion being capable of being positioned at least partially within the second vertebra or the spinal disc.

37. The system of claim 1,
wherein for at least a majority of the plurality of supports the support height for a central portion of the support is greater than the support height for a peripheral portion of the support.

38. The system of claim 1,
wherein the support height differs among the plurality of supports.

39. The system of claim 1,
wherein at least one of the plurality of supports includes a ridge at the first end or at the second end.

40. The system of claim 1, wherein the means for linking is at least partially insertable within a hole or a groove in the plurality of supports.

41. The system of claim 1, wherein the means for linking is attached at a central surface of the plurality of supports.

42. The system of claim 1, wherein the means for linking is attached at a lateral surface of the plurality of supports.

43. The system of claim 1, wherein the means for linking is attached at a peripheral surface of the plurality of supports.

44. The system of claim 1, wherein the means for linking is attached at the support first end or the support second end.

45. The system of claim 1, wherein the means for linking surrounds the plurality of supports.

46. The system of claim 1, wherein the means for linking comprises an extension of the plurality of supports.

47. The system of claim 1, wherein the means for linking comprises a hinge.

48. The system of claim 1, wherein the means for linking comprises a sheet.

49. The system of claim 48, wherein the sheet includes an opening.

50. The system of claim 1, wherein the means for linking comprises a stent.

51. The system of claim 1, wherein the means for linking comprises a mesh.

52. The system of claim 1, wherein the means for linking comprises an elongate member.

53. The system of claim 52, wherein the elongate member comprises a string.

54. The system of claim 52, wherein the elongate member comprises a wire.

55. The system of claim 52, wherein the elongate member comprises a rod.

56. The system of claim 1, wherein the means for linking comprises a spring.

57. The system of claim 56, wherein the spring comprises an arc.

58. The system of claim 56, wherein the spring comprises a helical coil.

59. A method for treating a spine, the spine including a first vertebra and a second vertebra, the first vertebra having a first endplate that is adjacent a spinal disc, the second vertebra having a second endplate that is adjacent the spinal disc, the first vertebra having a pedicle and a body wall, the method comprising:
(a) forming a channel that extends through the first vertebra, wherein the channel extends through the pedicle or the body wall and the channel extends through the first endplate, the channel having a channel axis and a channel diameter, the channel axis at the first endplate being oblique or perpendicular to the first endplate;
(b) providing an expandable implant, the expandable implant comprising a plurality of supports and means for linking the plurality of supports, the plurality of supports being capable of moving apart from one another, the plurality of supports having a support first end and a support second end and a support axis that extends from the support first end to the support second end, the plurality of supports having a support height, the expandable implant having an unexpanded diameter that is perpendicular to the support axis;
(c) advancing the expandable implant through the channel, wherein the unexpanded diameter is configured to permit passage of the expandable implant through the channel; and
(d) expanding the expandable implant to an expanded condition, wherein the expanding comprises moving the plurality of supports apart from one another, the expandable implant having an expanded diameter when the expandable implant is in the expanded condition, the expanded diameter being perpendicular to the support axis,
wherein the expanded diameter is configured to be greater than the channel diameter at the first endplate; and
wherein the support height is configured to permit the support second end to be positioned adjacent the second endplate while the support first end is positioned adjacent the first endplate while the support axis is oriented substantially perpendicular to the first endplate.

60. The method of claim 59,
wherein the channel extends through the pedicle and through the first endplate.

61. The method of claim 59,
wherein the forming causes the channel diameter for the central region to be greater than the channel diameter for a pedicle region of the channel and greater than the channel diameter for the endplate region of the channel.

62. The method of claim 59, further comprising:
preparing the spinal disc and the first endplate and the second endplate prior to advancing the expandable implant through the channel, wherein the preparing comprises removing at least a portion of a nucleus for the spinal disc and abrading the first endplate and abrading the second endplate.

63. The method of claim 59,
wherein the expanding further comprises inserting a wedge between the plurality of supports.

64. The method of claim 59,
wherein the expanding further comprises inflating a balloon that is positioned between the plurality of supports.

65. The method of claim 59,
wherein the expanding further comprises introducing bone graft material through a catheter into the expandable implant, the bone graft material being morselized or flowable, the bone graft material comprising bone or bone graft substitute.

66. The method of claim 59, further comprising:
introducing bone graft material between the first endplate and the second endplate using a catheter, the bone graft material being morselized or flowable, the bone graft material comprising bone or bone graft substitute.

67. The method of claim 59, further comprising:
introducing bone graft material through a catheter into the expandable implant when the expandable implant is in the expanded condition, the bone graft material being morselized or flowable, the bone graft material comprising bone or bone graft substitute.

68. The method of claim 59, further comprising:
inserting a central element between the plurality of supports when the expandable implant is in the expanded condition, the central element having a central element diameter that is configured to permit passage of the central element through the channel.

69. The method of claim 68, further comprising:
anchoring the central element in the first vertebra or the second vertebra.

70. The method of claim 59,
wherein the providing further comprises providing a second expandable implant, and wherein the method further comprises:
advancing the second expandable implant through the channel;
inserting the second expandable implant between the plurality of supports for the expandable implant when the expandable implant is in the expanded condition; and expanding the second expandable implant.

71. A method for treating a spine, the spine including a first vertebra and a second vertebra, the first vertebra having a first endplate that is adjacent a spinal disc, the second vertebra having a second endplate that is adjacent the spinal disc, the first vertebra having a body and a pedicle, the method comprising:
(a) forming a channel that extends through the first vertebra, wherein the channel extends through the pedicle and through the first endplate, the channel having a channel diameter, the channel having a pedicle region, a central region, and an endplate region,
wherein the channel diameter for the central region is greater than the channel diameter for the pedicle region and the channel diameter for the central region is greater than the channel diameter for the endplate region;
(b) providing an implant, the implant having an implant diameter, wherein the implant diameter is configured to permit passage of the implant through the pedicle region and through the endplate region;
(c) introducing the implant into the pedicle region; and
(d) advancing the implant through the channel, wherein at least a portion of the implant advances at least to the first endplate.

72. The method of claim 71, further comprising:
installing the implant, wherein the installing comprises positioning the implant at least partially within the spinal disc or at least partially within the first vertebra or at least partially within the second vertebra.

73. The method of claim 71,
wherein the forming comprises:
creating a predecessor channel that extends through the pedicle and through the first endplate, wherein the predecessor channel is coaxial with the channel in at least a portion of the pedicle region and the predecessor channel is coaxial with the channel in at least a portion of the endplate region; and
enlarging the central region for the predecessor channel,
wherein the enlarging causes the channel diameter for the central region to be greater than the channel diameter for the pedicle region and the enlarging causes the channel diameter for the central region to be greater than the channel diameter for the endplate region.

74. The method of claim 73,
wherein the enlarging comprises:
cutting or abrading the body where it surrounds the central region of the predecessor channel using a drill, the drill comprising a steerable drill or a flexible drill, the drill comprising a retractable cutting head and a sheath, the retractable cutting head being capable of retracting within the sheath, the sheath dimensioned to be insertable within the predecessor channel, the retractable cutting head capable of emerging from a distal end of the sheath, wherein a cutting head radius for the emerged retractable cutting head is greater than half of the channel diameter for the pedicle region.

75. The method of claim 73,
wherein the enlarging comprises:
advancing a dilator in the predecessor channel to a position within the central region; and
dilating the dilator for displacing cancellous bone of the body that surrounds the central region of the predecessor channel.

76. The method of claim 75,
wherein the dilator comprises a balloon and an inflation line that is connected to the balloon, and wherein the dilating comprises inflating the balloon.

77. The method of claim 75,
wherein the dilator comprises a wedge.

78. The method of claim 71,
wherein the forming comprises:
creating a first predecessor channel and a second predecessor channel, wherein the second predecessor channel diverges from the first predecessor channel in at least a portion of the central region.

79. A system as in any one of claim 1, 2, 3, 4, 5 or 6, wherein the expandable implant includes a passage for a guidewire, the passage extending from the support first end to the support second end.

80. A system as in any one of claim 1, 2, 3, 4, 5 or 6, further comprising means for expanding the expandable implant.

81. A system as in any one of claim 1, 2, 3, 4, 5 or 6, wherein the expandable implant further comprises a central element that is insertable between the plurality of supports when the expandable implant is in the expanded condition, the central element having a central element diameter that is configured to permit passage of the central element through the channel.

82. The system of claim 81, wherein the central element includes a wall and a lumen.

83. The system of claim 81, wherein the central element includes means for anchoring in the first vertebra or the second vertebra.

84. The system of claim 81, wherein the central element comprises a first central element and a second central element, wherein the first central element is positioned adjacent the support first end and the second central element is positioned adjacent the support second end.

85. A system as in any one of claim 1, 2, 3, 4, 5 or 6, wherein the expandable implant comprises a first expandable implant and a second expandable implant, and wherein the second expandable implant is insertable between the plurality of supports for the first expandable implant when the first expandable implant is in the expanded condition.

86. The system of claim 85, wherein at least a majority of the plurality of supports for the second expandable implant are dimensioned to be insertable into a plurality of gaps between the plurality of supports for the first expandable implant in the expanded condition.

87. A system as in any one of claim 1, 2, 3, 4, 5 or 6,
wherein the expandable implant includes a passage for a guidewire, the passage extending from the support first end to the support second end; and
wherein at least one of the plurality of supports has a central surface that is at least partially curved relative to the support axis, the at least partially curved central surface defining at least a portion of the passage for the guidewire.

88. A system as in any one of claim 1, 2, 3, 4, 5 or 6, further comprising a guidewire,
wherein a first flexibility for a central portion of the guidewire is greater than a second flexibility for a distal portion of the guidewire, the distal portion being capable of being positioned at least partially within the second vertebra or the spinal disc.

89. A system as in any one of claim 1, 2, 3, 4, 5 or 6, wherein for at least a majority of the plurality of supports the support height for a central portion of the support is greater than the support height for a peripheral portion of the support.
